(19) United States
(12) Patent Application Publication
(10) Pub. No.: US 2025/0114131 A1
(43) Pub. Date: Apr. 10, 2025

(54) DISTAL RADIUS IMPLANTS AND INSTRUMENTS

(71) Applicant: GLOBUS MEDICAL, INC., Audubon, PA (US)

(72) Inventors: Megan Duman, Horsham, PA (US); Stephanie Wolfe, Perkasie, PA (US); Brendan Inglis, Phoenixville, PA (US); Jesse Rush, Wayne, PA (US); Richard Scheinfield, Philadelphia, PA (US); Garret Norton, Lima, OH (US); Jessica Sandoe, Blue Bell, PA (US)

(21) Appl. No.: 18/530,799

(22) Filed: Dec. 6, 2023

Related U.S. Application Data
(60) Provisional application No. 63/588,165, filed on Oct. 5, 2023.

(51) Int. Cl. A61B 17/80

(52) U.S. Cl. CPC A61B 17/8057; A61B 17/8061

(57) ABSTRACT

Devices, systems, instruments, and methods for promoting healing and stability for bone fractures. The bone stabilization system may include a variety trauma plates and one or more bone fasteners configured to secure the plate to bone. The bone plates may be used for the fixation of fractures and fragments in the distal radius. The distal radius plating may be used to create a rigid construct with permanent fixation to promote primary healing and stability.

### CROSS-REFERENCE TO RELATED APPLICATIONS

This application is a non-provisional application that claims priority to provisional application 63/588,165 dated Oct. 5, 2023, which is incorporated in its entirety herein.

### FIELD OF THE INVENTION

The present disclosure relates to surgical devices, and more particularly, to stabilization systems, for example, for trauma applications.

### BACKGROUND OF THE INVENTION

Distal radius plates may be used by orthopedic surgeons as an internal fixation device for a variety of fracture patterns in the volar region of the distal radius, commonly known as wrist fractures. These plates may be placed on the volar aspect (palm-side) of the distal radius and are configured to stabilize the fractured bone and allow it to heal in the correct anatomical position. Typical indications may include buttressing of comminuted/multi-fragmentary fractures, metaphyseal and diaphyseal fractures, intra-articular and extra-articular distal radius fractures, fractures in osteopenic bone, and non-unions and malunions.

A volar distal radius plate may include a thin, lightweight implant that is designed for minimal soft tissue disruption. These implants need to be strong enough to withstand everyday forces. Distal radius volar plates that are too thick or sit up too close to the Watershed line (an approximate line that is the highest point on the most distal, volar edge of the radius that signifies a point proximal to the articular surface where ligaments begin to transition from the radius to the joint and hand) can cause soft tissue irritation and, in severe cases, tendon ruptures. These soft tissue injuries may lead to revision surgeries and the potential of further complications. Thus, there remains a need for improved plate styles and plating systems for the fixation of fractures and fragments of the distal radius.

### SUMMARY OF THE INVENTION

To meet this and other needs, and in view of its purposes, the present application provides devices, systems, instruments, and methods for promoting healing and stability for bone fractures. In particular, the plates may include a comprehensive offering of plate styles able to treat a vast array of fracture patterns. The plates may include shallow-contour implants, which minimize soft tissue irritation in the volar region and reduce the likelihood of tendon ruptures or other potential complications.

According to one embodiment, a bone stabilization system includes a collection of trauma plates configured for the fixation of fractures and fragments of the distal radius. Each bone plate is configured to be positioned against an exterior surface of the distal radius. The system includes one or more fasteners, such as locking and/or non-locking bone screws that a surgeon may select based on preference for a specific anatomical case. The locking fasteners may connect to the plate and the bone to thereby lock the plate to the distal radius. The non-locking fasteners may be able to position and center the plate through a positioning slot. The plate may include one or more guide wire or K-wire holes configured to help guide and temporarily hold the plates in position. The plates may include double row and single row polyaxial volar plates, double row and single row monoaxial volar plates, diaphyseal-metaphyseal plates, and flexor pollicis longus (FPL) tendon volar plates.

According to one embodiment, a distal radius bone plate includes a body extending along a central longitudinal axis from a proximal end configured to sit on a shaft of a radius to a distal end configured to sit on a distal radius, a top surface and an opposite, bottom surface configured to contact the radius, and an elongated proximal shaft and an enlarged distal head extending therefrom. The enlarged distal head defines a tendon groove in the distal end configured to accommodate a flexor pollicus longus tendon. The tendon groove includes a concave recess at a distal edge of the plate forming two rounded lobes on either side of the groove. The lobes include an ulnar-side lobe and a radial-side lobe.

The distal radius bone plate may include one or more of the following features. The two rounded lobes may be non-symmetrical, for example, about the central longitudinal axis. The ulnar-side lobe may be longer or otherwise of a different shape than the radial side lobe. The tendon groove may be located off-center of the central longitudinal axis of the plate. The radial-side lobe may be thicker than the ulnar-side lobe. The ulnar-side lobe may include a curved ulnar surface on the bottom surface of the plate to promote best fit of the distal head with the distal radius. The ulnar-side lobe may include a distal ulnar undercut through a distal-most portion of the ulnar-side lobe. The ulnar-side lobe may include a variable chamfered surface along its edge to minimize tendon disruption.

According to one embodiment, a stabilization system for stabilizing a distal radius includes a plate and a plurality of fasteners. The plate has a top surface and an opposite, bottom surface configured to contact a radius. The plate has an elongated proximal shaft and an enlarged distal head extending therefrom. The distal head defines a tendon groove configured to receive a flexor pollicus longus tendon. The tendon groove includes a concave recess at a distal edge of the plate forming two rounded lobes on either side of the groove. The lobes include an ulnar-side lobe and a radial-side lobe each including fastener openings. The ulnar-side lobe is longer than the radial side lobe. The plurality of fasteners are receivable through the fastener openings and configured to lock the plate to the distal radius.

The stabilization system may include one or more of the following features. The fastener openings may include polyaxial openings having a cone of angulation up to 40 degrees. The radial-side lobe may define a first opening with a trajectory targeted at a radial styloid. The lobes may define a distal-most row of fastener openings, and when the fasteners are received therein, screw trajectories of the distal-most row of fastener openings may be aligned so that nominal trajectories of the fasteners follow articular surfaces of a radiocarpal joint and a distal radio-ulnar joint. The lobes may define a second row of fastener openings, and when the fasteners are received therein, screw trajectories of the second row of fastener openings may have trajectories that fit between the distal-most row of fasteners such that the trajectories converge with the distal-most row screw trajectories. The second row of fastener openings may include a first opening in the radial-side lobe and a second fastener opening in the ulnar-side lobe, the first opening receives a fastener that extends dorsally and toward the radial styloid screw trajectory and the second opening receive a fastener that extends dorsally and fits between trajectories of the distal-most openings on the ulnar-side lobe. The proximal shaft may define a positioning slot configured to receive a non-locking fastener such that the plate is adjustable in proximal-distal and/or medial-lateral directions during provisional placement of the plate.

According to one embodiment, a method of installing a distal radius plate includes one or more of the following steps in any suitable order: (a) providing a distal radius plate having an elongated proximal shaft and an enlarged distal head extending therefrom, the distal head defines a tendon groove having a concave recess at a distal edge of the plate forming two rounded lobes on either side of the groove, the lobes include an ulnar-side lobe and a radial-side lobe each defining fastener openings and guide wire openings therethrough, the proximal shaft defines a positioning slot, fastener openings, and guide wire openings therethrough; (b) provisionally placing the plate against a distal radius by inserting a non-locking screw through the positioning slot and moving the plate in proximal-distal and/or medial-lateral directions to center the plate on the distal radius; (c) inserting one or more guide wires through the guide wire openings to hold the plate in position and/or use as a guide for inserting fasteners; and (d) inserting fasteners through the fastener openings in the radial-side lobe including one fastener into a radial styloid and one fastener following a radiocarpal joint, and in the ulnar-side lobe including one fastener following the radiocarpal joint and one fastener following a distal radio-ulnar joint. The plate may define a second row of fastener openings, and the method may further include (c) inserting fasteners through the second row of fastener openings including one fastener in the radial-side lobe that extends dorsally toward the radial styloid fastener and one fastener in the ulnar-side lobe that extends dorsally and fits between the radiocarpal joint and distal radio-ulnar joint fasteners in the ulnar-side lobe. The method may also include (f) independently adjusting the radial-side and ulnar-side lobes intraoperatively to optimize placement against the distal radius. The fastener openings may be polyaxial holes such that the fasteners are variable angle fasteners providing for variable angle screw insertion with up to 40 degrees of cone of angulation. Alternatively, the fastener openings may be monoaxial holes such that the fasteners are fixed angle fasteners providing for fixed angle screw insertion.

According to one embodiment, an instrument set for installing the distal radius plates may include a drill bit, a monoaxial locking drill guide, a calibrated measuring block, and a calibrated polyaxial drill guide. The drill bit includes a shaft with cutting flutes and the shaft has a series of markings. The monoaxial locking drill guide is configured to lock into monoaxial holes in a plate. The monoaxial locking drill guide includes a cylindrical body with a central cannulation. The calibrated measuring block is configured to provide a calibrated measurement when drilling holes through a monoaxial volar targeting guide. The calibrated measuring block has a cannulated body with a cylindrical distal tip. The calibrated polyaxial drill guide is configured to measure drilled depths for monaxial and polyaxial trajectories in plate holes. The calibrated polyaxial drill guide has a polyaxial measuring block and a monoaxial measuring block on opposite ends of a handle. The series of markings on the drill bit align with the monoaxial locking drill guide, calibrated measuring block, and the polyaxial and monoaxial measuring blocks on the calibrated polyaxial drill guide to determine a screw size for a given drilled hole.

The instrument set may include one or more of the following features. The series of marking on the drill bit may include a primary proximal laser etch configured to align with the calibrated measuring block and polyaxial and monoaxial measuring blocks. The series of marking on the drill bit may include distal secondary laser etches at equal intervals configured to align with the monoaxial locking drill guide. The monoaxial locking drill guide may define a counterbore, a threaded recess, a drive recess, and a drill cannulation along a central tool axis. The instrument set may also include a K-wire sleeve with a threaded distal end, and the counterbore and the threaded recess in a top of the monoaxial locking drill guide may facilitate a connection with the threaded K-wire sleeve. The calibrated measuring block may have a rectangular block face with an enlarged grip. The calibrated measuring block may have a viewing window with calibrated etches surrounding the window. When the drill bit is positioned through the calibrated measuring block, one of the markings on the drill bit may align with the calibrated etches to indicate the screw size for the given drilled hole. The polyaxial measuring block may have a polyaxial tip that provide 20 degrees of polyaxial positioning in the plate holes. The monoaxial measuring block may have a nominal angle tip that provides a zero degree nominal trajectory in the plate holes. The polyaxial and monoaxial measuring blocks may include a viewing window with calibrated etches surrounding the window. When the drill bit is positioned through the calibrated measuring block, one of the markings on the drill bit may align with the calibrated etches to indicate the screw size for the given drilled hole. The instrument set may also include monoaxial and polyaxial volar targeting guides configured to attach to respective plates to drill pilot holes at appropriate trajectories for each plate hole.

According to one embodiment, a targeting guide system includes a volar distal radius plate and a targeting guide with an attachment screw. The volar distal radius plate has an elongated proximal shaft and an enlarged distal head extending therefrom. The enlarged distal head defines a tendon groove having a concave recess at a distal edge of the plate forming two rounded lobes on either side of the groove. The volar distal radius plate defines a plurality of fastener openings, guide wire holes, and an attachment opening. The targeting guide is configured to be disposed on the distal head of the volar distal radius plate and has a plurality of cannulated openings corresponding to the respective fastener openings, and a retaining hole corresponding to the attachment opening. The attachment screw is threaded through the retaining hole and into the attachment opening, thereby temporarily securing the targeting guide to the volar distal radius plate.

The targeting guide system may include one or more of the following features. The attachment screw may include a knurled elongated head with a partially threaded shaft. The retaining hole may include an upper counterbore, a threaded section, and a lower counter bore. The upper counterbore may create a parallel mating surface for a bottom of the knurled elongated head when fully seated therein. The attachment opening may be located on an ulnar side of the volar distal radius plate adjacent to a graft window. The attachment opening may define a female thread configured to receive a corresponding male thread on the attachment screw. The targeting guide may include guide wire openings corresponding to the respective guide wire holes in the volar distal radius plate, and some of the cannulated openings and guide wire openings in the targeting guide may overlap. The targeting guide may include a guide wire alignment boss and a graft window cam extending from a bottom surface of the targeting guide, which prevent rotation of the targeting guide relative to the volar distal radius plate.

Also provided are kits for the stabilization systems including bone plates of varying types and sizes, fasteners of varying types and sizes including locking fasteners, non-locking fasteners, compression fasteners, polyaxial fasteners, fixed angle fasteners, or any other suitable fasteners, drill guides, K-wires, sutures, instruments, and other components for installing the same.

### BRIEF DESCRIPTION OF THE DRAWINGS

A more complete understanding of the present invention, and the attendant advantages and features thereof, will be more readily understood by reference to the following detailed description when considered in conjunction with the accompanying drawings, wherein:

FIG. 1 shows a collection of plate styles configured for fixation of fractures of the volar distal radius;

FIG. 2 shows a collection of double row polyaxial volar plate styles;

FIGS. 3A-3B show a distal radius double row volar plate stabilization system with screw trajectories according to one embodiment;

FIG. 4 shows a double row polyaxial volar plate according to one embodiment;

FIGS. 5A-5B show a side view and distal end view, respectively, of the double row polyaxial volar plate;

FIG. 6 shows a collection of monoaxial double row volar plate styles;

FIGS. 7A-7B show a distal radius monoaxial double row plate stabilization system with screw trajectories according to one embodiment;

FIG. 8 shows a monoaxial double row volar plate according to one embodiment;

FIGS. 9A-9B show side views of the monoaxial double row plate;

FIGS. 10A-10B show a distal contour and ulnar undercut on the monoaxial double row plate to promote best fit on the ulnar side of the plate;

FIG. 11 shows a monoaxial stacked non-locking hole according to one embodiment;

FIGS. 12A-12B show a monoaxial double row volar plate according to one embodiment;

FIGS. 13A-13C show a top view, ulnar side view, and radial side view, respectively, the monoaxial double row volar plate system with screw trajectories according to one embodiment;

FIG. 14 shows a collection of single row polyaxial volar plate styles;

FIG. 15 shows a single row volar plate according to one embodiment;

FIGS. 16A-16B show a side view and distal end view, respectively of the single row volar plate;

FIG. 17 shows a monoaxial single row volar plate according to one embodiment;

FIGS. 18A-18B show a side view and radial side view of the monoaxial single row volar plate;

FIG. 19 shows a collection of diaphyseal-metaphyseal plates configured to sit on the volar face of the radius;

FIG. 20 shows a diaphyseal-metaphyseal plate according to one embodiment;

FIG. 21 shows a side view of the diaphyseal-metaphyseal plate;

FIG. 22A-22C show a top view, ulnar side view, and radial side view, respectively, of a monoaxial flexor pollicis longus (FPL) volar plate system with screw trajectories according to one embodiment;

FIGS. 23A-23B show a monoaxial FPL volar plate according to one embodiment;

FIGS. 24A-24D show an example of a short merged hole with a locking portion and a non-locking portion;

FIGS. 25A-25B show perspective and cross-sectional views, respectively, for a locking screw inserted through the short merged hole at a nominal trajectory;

FIGS. 26A-26B show perspective and cross-sectional views, respectively, for a locking screw inserted through the short merged hole at a polyaxial trajectory;

FIGS. 27A-27B show perspective and cross-sectional views, respectively, for a non-locking screw inserted through the short merged hole at a nominal trajectory;

FIGS. 28A-28D show an example of an oblong merged hole with a locking portion and a non-locking portion;

FIGS. 29A-29B show perspective and cross-sectional views, respectively, for a locking screw inserted through the oblong merged hole at a nominal trajectory;

FIGS. 30A-30B show perspective and cross-sectional views, respectively, for a locking screw inserted through the oblong merged hole at a polyaxial trajectory;

FIGS. 31A-31C show perspective and cross-sectional views, respectively, for a non-locking screw inserted through the oblong merged hole, which is permitted to translate along the oblong portion of the hole;

FIG. 32 shows a kit including instruments for installing the distal radius plates;

FIG. 33 shows a diaphyseal metaphyseal plate trial according to one embodiment;

FIGS. 34A-34B show a perspective view and cross-sectional view, respectively, of a monoaxial locking drill guide according to one embodiment;

FIGS. 35A-35C show perspective, side, and cross-sectional views, respectively, of a K-wire sleeve according to one embodiment;

FIG. 36 shows a cross section of the attachment between the K-wire sleeve and the locking drill guide according to one embodiment;

FIGS. 37A-37B show a measuring block for a monoaxial volar targeting guide according to one embodiment;

FIGS. 38A-38B show a calibrated polyaxial drill guide including a close-up of the scalloped and contoured handle according to one embodiment;

FIGS. 39A-39B show the calibrated measuring blocks of the polyaxial drill guide including the calibrated markings with different tips;

FIGS. 40A-40B show a calibrated drill bit according to one embodiment;

FIG. 41 shows a kit for a one drill concept with components providing alternative measurement options;

FIG. 42 shows a collection of polyaxial volar targeting guides;

FIG. 43 shows an attachment mechanism for the volar targeting guides according to one embodiment;

FIG. 44 shows a collection of monoaxial volar targeting guides;

FIG. 45 shows an attachment mechanism for the volar targeting guides according to one embodiment;

FIGS. 46A-46B show an alignment boss and graft window for the targeting guide according to one embodiment;

FIG. 47 shows a volar targeting guide attached to an implant according to one embodiment;

FIGS. 48A-48B show a top view and side view, respectively, of a volar targeting guide with drill guides, K-wire holes, alignment boss, retaining hole, and graft window cam according to one embodiment;

FIGS. 49A-49B show a cross-section of the retaining hole and a side view of an attachment screw for securing the targeting guide to the plate according to one embodiment; and

FIGS. 50A-50B show a cross-section of the assembly and a close-up view of the attachment screw interfacing with the retaining hole and plate according to one embodiment.

### DETAILED DESCRIPTION OF THE INVENTION

Embodiments of the disclosure are generally directed to devices, systems, instruments, and methods for promoting healing and stability for bone fractures. The plates may include a comprehensive offering of implants or plate styles configured for the fixation of fractures and bone fragments. The bone plates may be used to create a very rigid construct with permanent fixation to promote primary healing and stability. Alternatively, the plates are also capable of being used as temporary or supplemental fixation.

A series of trauma plates may be used for the fixation of fractures and fragments in the distal radius. The forearm is made up of two bones: the radius and ulna. The radius is located on the thumb side of the arm. The end of the radius connected to the wrist joint is called the distal radius. The volar aspect of the distal radius is the palm-side of the bone. When the radius breaks near the wrist, it is called a distal radius fracture. The distal radius plates may be used to address simple and complex fractures in the distal radius to ensure proper alignment and facilitating healing.

The volar distal radius plates include thin, strong, lightweight implants configured for minimal soft tissue disruption. The distal radius volar plates may include low profile shallow contoured implants that sit farther away from the Watershed line (an approximate line that is the highest point on the most distal, volar edge of the radius that signifies a point proximal to the articular surface where ligaments begin to transition from the radius to the joint and hand), thereby allowing for less soft tissue and/or tendon irritation.

Although the plates are generally described with reference to stabilizing the radius, it will be appreciated that the stabilization systems described herein may be used or adapted to be used for the fixation of other areas or other bones as well including the femur, tibia, humerus, clavicle, fibula, ulna, bones of the hand, bones of the feet, or other suitable bone(s) or joint(s). The bone plates 10 may be available in a variety of lengths, widths, and styles based on the anatomy of the patient and types of fractures. The systems may be adapted to secure small or large bone fragments, single or multiple bone fragments, or otherwise secure one or more fractures or joints.

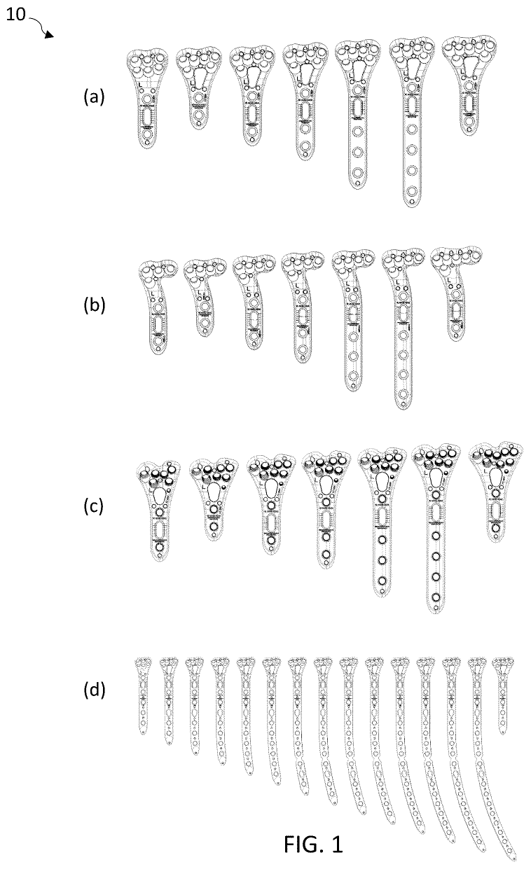

Turning now to the drawing, where like reference numerals refer to like elements, FIG. 1 shows a collection of plate styles 10 configured for fixation of fractures and fragments of the volar distal radius. Several different plate styles 10 may be used in the treatment of various fractures of the distal radius including: (a) double row polyaxial volar plates; (b) single row polyaxial volar plates; (c) double row monoaxial plates; and (d) diaphyseal-metaphyseal plates. Additional plate styles 10 may include flexor pollicis longus (FPL) tendon volar plates, and single and double row monoaxial volar plates. The volar distal radius plates 10 may be provided in a series of lengths and widths, as well as one and two row options to accommodate the volar distal radius.

These plate styles 10 are configured to stabilize the fractured bone, thereby allowing the bone to heal in the correct anatomical position. The bone plate 10 spans the bone fracture(s) to hold the bone fragments together, allowing the bone to heal in the correct alignment. These plates 10 may be provided in a number of variations in a surgical tray, which include for example various types, sizes, and configurations. The tray selection may allow for the surgeon to select a desired plate during surgery after opening the wound area and considering the plating needs for the patient.

Each plate 10 may be configured to receive one or more bone fasteners 12. The fasteners 12 may include locking fasteners, non-locking fasteners, or any other fasteners known in the art. The fasteners 12 may comprise bone screws or the like. The fasteners 12 may be cannulated such that they may be guided into place over guide wires. The fasteners 12 may also include other fasteners or anchors configured to be secured or engaged with bone, such as nails, spikes, staples, pegs, barbs, hooks, or the like. In some embodiments, the fasteners 12 may include fixed and/or variable angle bone screws. The fastener 12 may include a head portion and a threaded shaft portion configured to engage bone. In the case of a locking fastener 12, the head portion may include a textured area, such as threads, around its outer surface sized and configured to engage with an opening 16, for example, with corresponding threads or textured area in the opening 16 in order to lock the fastener 12 to the plate 10. In the alternative, for a non-locking fastener 12, the head portion may be substantially smooth and rounded to allow for plate positioning and/or dynamic compression of the bone 2. The fasteners 12 may have a threaded shaft portion configured to secure the plate 10 and fastener 12 to bone.

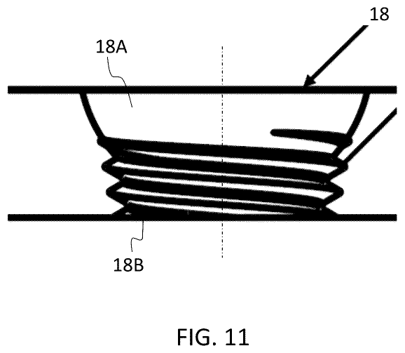

The plates 10 may include one or more openings or hole types. The fastener openings 14, 16, 18 may include cylindrical openings, conical openings, elongated openings, threaded openings, textured openings, non-threaded and/or non-textured openings, and the like. The openings 14, 16, 18 extending through the plate 10 are configured to accept locking fasteners, non-locking fasteners, or a combination of both locking and non-locking fasteners that are able to position the plate and/or affix the plate 10 to the bone. For example, a first fastener opening type may include an elongated opening, positioning slot, or sliding slot 14, which allows for static insertion of non-locking screws 12 into the bone and/or plate positioning along the bone. A second fastener opening type may include a polyaxial locking hole 16 with a textured portion configured to engage a head portion of the locking fastener. The polyaxial locking screw 12 may include threads or a textured area configured to deform and/or engage with the locking hole 16, thereby locking the fastener 12 to the plate 10. The polyaxial locking may provide for variable angle screw insertion, for example, with up to 40 degrees of cone of angulation. A third fastener opening type may include a monoaxial hole 18 configured to engage a head portion of a fixed angle locking fastener. The monoaxial hole 18 may include locking holes, which are locked to the plate 10 only in one designated direction, or non-locking holes, such as non-locking stacked holes (as shown in FIG. 11). Additional details on these and other types of openings are provided in further detail in U.S. Pat. No. 11,432,857, which is incorporated by reference herein in its entirety for all purposes.

In addition to fastener openings, a fourth opening type may include a guide wire or K-wire hole 20, which is configured to receive a guide wire or K-wire therethrough. The guide wire or K-wire holes 20 have a diameter smaller than the diameter of the fastener openings 16, 18 and may be sized and dimensioned to receive the guide wire or K-wire. A fifth opening type may include a graft retaining opening or bone graft window 50 configured to receive bone graft or other suitable bone growth enhancing material. A sixth opening type may include an attachment opening or hole 52 configured to attach to an instrument, such as a targeting guide. The plates 10 may comprise any suitable number of openings in any suitable configuration. These openings allow surgeons flexibility for fastener placement based on preference, anatomy, and fracture location.

The bone plates 10 may be comprised of titanium, stainless steel, cobalt chrome, carbon composite, plastic or polymer-such as polyetheretherketone (PEEK), polyethylene, ultra high molecular weight polyethylene (UHMWPE), resorbable polylactic acid (PLA), polyglycolic acid (PGA), combinations or alloys of such materials or any other appropriate material that has sufficient strength to be secured to and hold bone, while also having sufficient biocompatibility to be implanted into a body. Similarly, the fasteners 12 may be comprised of titanium, cobalt chrome, cobalt-chrome-molybdenum, stainless steel, tungsten carbide, combinations or alloys of such materials or other appropriate biocompatible materials. Although the above list of materials includes many typical materials out of which bone plates and bone fasteners are made, it should be understood that bone plates and fasteners comprised of any appropriate material are contemplated.

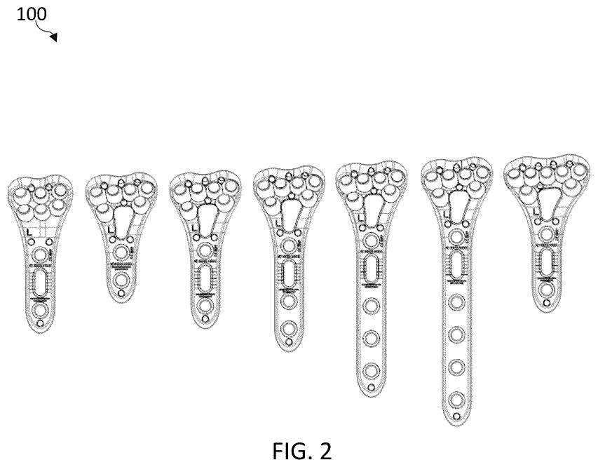

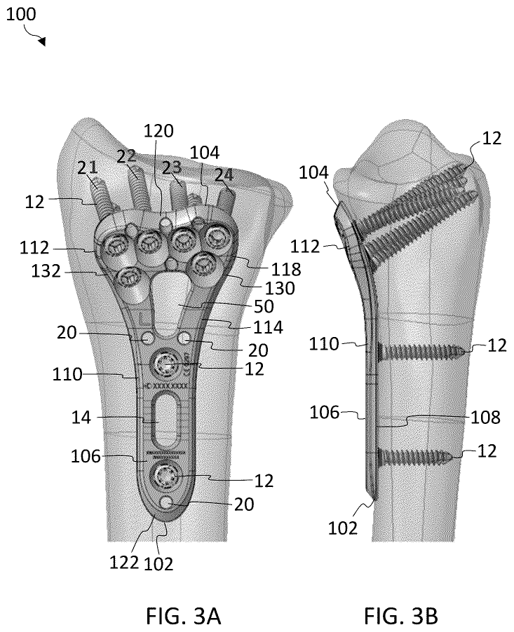

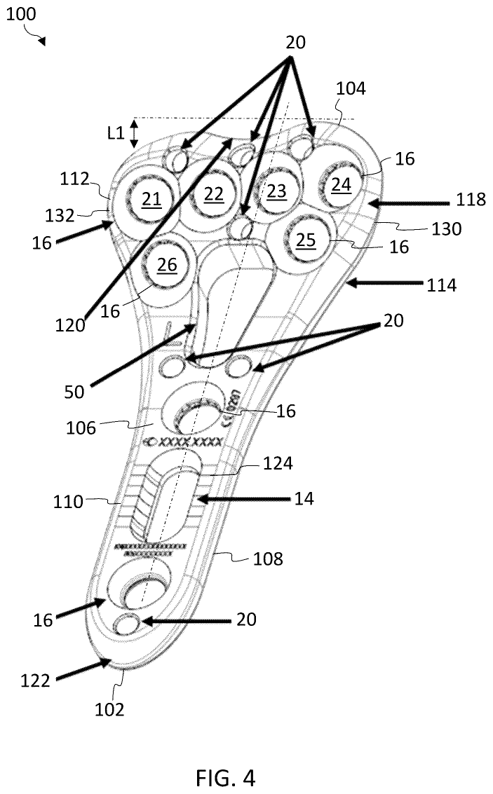

Turning now to FIGS. 2-5B, a collection of double row polyaxial volar plate styles 100 is shown. FIGS. 3A-3B depict the anatomy of the distal radius with a double row polyaxial volar implant 100 contoured to sit on the volar aspect of the distal radius. The double row polyaxial volar implant 100 is configured to be positioned against an outside face of the bone, for example, of the distal radius, and secured with fasteners 12. The double row polyaxial volar plate 100 has a body that extends along a central longitudinal axis from a first end or proximal end 102 configured to sit on the shaft of the radius to a second end or distal end 104 configured to sit on the distal end of the radius. The plates 100 are configured in both left and right designs, in a mirrored configuration, in order to address the anatomy of both the left and right arms of the patient.

The double row polyaxial volar plate 100 includes a top surface 106 and an opposite, bottom surface 108 configured to contact adjacent bone. The top and bottom surfaces 106, 108 are connected by opposite side surfaces extending from the first to second ends 102, 104 of the plate 100. The plate 100 includes a proximal shaft 110 and an enlarged distal head 112. The proximal shaft 110 may have an elongated longitudinal body, having a length greater than its width, extending along the central longitudinal axis of the plate 100. The proximal shaft 110 is configured to contact the shaft of the radius. The distal head 112 is configured to contact the distal end of the radius. The enlarged distal head 112 has a width greater than the width of the proximal shaft 110. The enlarged distal head 112 may gradually flare out in width from the proximal shaft 110. The distal head 112 includes an ulnar side 130 configured to contact the ulnar portion of the radius and a radial side 132 configured to contact the portion of the radius toward the radial styloid.

The double row polyaxial volar plate 100 may include two struts of material running up both the radial and intermediate columns of the bone with window 50 defined in between the two struts. The window 50 may be configured to receive bone graft or other suitable bone growth enhancing material. In addition, the window 50 may provide visualization of the plate 100 with respect to the radius in the operating environment and on imaging (e.g., fluoroscopy). The window 50 may be an asymmetrical shape or, if present, may be of any suitable shape, size, and dimension.

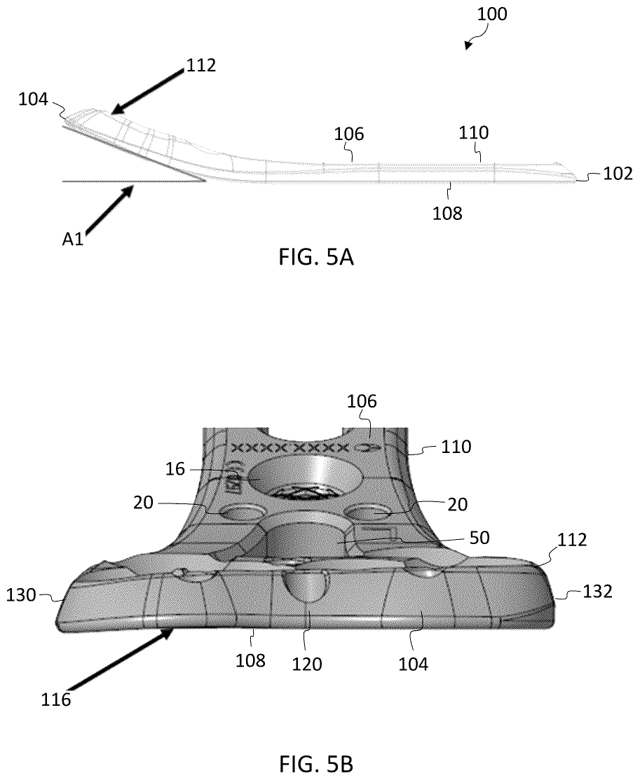

As best seen in FIG. 5A, the bottom 108 of the plate 100 may have a pre-contoured geometry. In particular, the bottom surface 108 of the plate 100 may include an anatomic contour configured to follow the best approximation of average distal radius anatomy, flaring up at the distal head 112. The distal plate thickness is optimized at the distal head 112 to sit low and have a generally low profile. For example, the thickness of the plate 100 may be held at 2 mm along the shaft 110 and tapers to a thickness of 2.75 mm along the distal head 112 of the plate 100. The distal head 112 may have a distal angulation angle A1 relative to the shaft 110 for an optimized plate contour. For example, the volar plates 100 may have a distal angulation A1 of about 24° in order have the plate 100 sit lower on the radius and not infringe on the Watershed line, which defines the border between the radiocarpal (RC) joint and the volar surface of the bone. As best seen in FIG. 5B, a curved ulnar surface 116 may be provided on the ulnar side 130 to promote best fit of the distal head 112 with the distal radius. The distal edge contour is a curved surface 116 configured to facilitate best fit of the contour on the ulnar side 130 of the radius 116. The curved ulnar surface 116 may have a radius, for example, of about 30 mm or other suitable dimension to fit the radius of the patient.

The edges 114 of plate 100 may be chamfered, for example, with a variable chamfered surface 118 to minimize tendon disruption. The variable chamfered surface 118 may include a beveled edge that changes in angle or width across its length. The significant chamfer 118 on the distal end 104 of the plate 100 may help to ensure minimal tendon disruption (specifically of the flexor pollicus longus and flexor carpi radialis) by maintaining a low plate profile over the relevant tendon sites. The variable chamfered surface 118 may extend along the ulnar side 130 of the plate 100 to reduce tendon irritation.

The distal head 112 of plate 100 may define a flexor pollicus longus (FPL) tendon groove 120. The tendon groove 120 may include a shallow concave recess in the distal edge 114 of the plate 100 forming two rounded lobes 130, 132 on either side of the shallow groove 120. The rounded lobes form an ulnar side lobe 130 and a radial side lobe 132. The two rounded lobes 130, 132 may be non-symmetrical, for example, about the central longitudinal axis with one lobe 130, 132 being longer, wider, thicker/thinner and/or differently shaped than the other 130, 132. The tendon groove 120 may be located off-center of the central longitudinal axis of the plate 100. In one embodiment, the ulnar-side lobe 130 may have a greater length L1 and is longer or taller than the radial side lobe 132. In this manner, the ulnar side lobe 130 may have a greater or larger degree of curvature than the radial side lobe 132. The tendon groove 120 provides a smooth transition between bone and plate 100 for the flexor pollicis longus tendon to run over. The tendon groove 120 may help to prevent tendon irritation and reduce the likelihood of tendon ruptures.

The proximal end 102 of the shaft 110 may define a tapered tip 122. The proximal shaft 110 may terminate with a taper such that it has a width and/or thickness less than the remainder of the elongated shaft 110, thereby maintaining a low profile at the proximal end 120. The low-profile nature of the plate 100 reduces the risk of soft tissue irritation and minimizes protrusion, making it less perceptible under the skin and more comfortable for the patient.

The double row polyaxial volar plate 100 includes one or more through openings 14, 16 configured to receive one or more bone fasteners 12. In some embodiments, a single sliding slot 14 may be defined through the proximal shaft 110, which is configured to receive a non-locking screw 12. For the shortest plate length, the sliding slot 14 may be omitted. When present, the sliding slot 14 may be aligned with the central axis of the implant 100. The sliding slot 14 may permit proximal-distal (P-D) adjustment of the plate 100 during provisional placement of the plate 100. In some cases, the sliding slot 14 may be sized and dimensioned to allow for proximal-distal adjustment as well as medial-lateral (M-L) adjustment of the plate 100. This allows the user to optimally center the plate position along the shaft of the bone prior to locking screw insertion. One or more markings 124 may be provided in and around the slot 14 on the top surface 106 of the plate 100, which are configured to help the user optimally align the plate 100 on the radius.

The double row polyaxial volar plate 100 includes one or more polyaxial openings 16 configured to receive one or more locking bone fasteners 12. The distal radius double row volar plate 100 may define polyaxial holes 16 (e.g., 2.5 mm polyaxial locking holes) with a 40° cone of angulation to accommodate a wide variety of anatomy and fracture patterns. The proximal shaft 110 may define a series of proximal polyaxial holes 16 aligned with the central axis of the implant 100. When present, the sliding slot 14 may be aligned with and located between opposite polyaxial holes 16. Depending on the length of the plate 100, the plate 100 may define between two and five proximal polyaxial holes 16. For longer plates 100, the additional proximal polyaxial holes 16 may be located proximal to the sliding slot 14.

The distal head 112 may define a double row of distal polyaxial holes 16. The double row of distal polyaxial holes 16 may be divided between the ulnar-side and radial-side lobes 130, 132. The distal-most or first row of polyaxial holes 16 may include three to five polyaxial holes 16. The second row of polyaxial holes 16 may be seated beneath the first row. The second row of distal polyaxial holes 16 may include two or three polyaxial holes. The second row of distal polyaxial hole 16 may be located on opposite sides of the graft window 50, if present.

As best seen in FIG. 3A, the screw trajectories 21, 22, 23, 24 of the distal-most row of screws 16 may be aligned so that their nominal trajectories follow the articular surfaces of both the radiocarpal joint 21, 22, 23 and the distal radio-ulnar joint (DRUJ) 24. For example, the radial-side lobe 132 may define screw trajectories 21, 22 and the ulnar-side lobe 130 may define screw trajectories 23, 24. This allows the distal-most row of screws 12 to buttress and support the articular surfaces during fracture reconstruction. The radial styloid screw trajectory 21 provides the severe angle necessary to reach the very tip of the radial styloid.

For applicable plates 100, the second row of distal screws 12 may have trajectories 25, 26 that fit between the distal-most row of screws 12 such that the trajectories 25, 26 converge with the first row screw trajectories 21, 22, 23, 24. The ulnar-side lobe 130 may define screw trajectory 25 and the radial side lobe 13 may define screw trajectory 26. For example, the second row screws 12 may include ulnar side trajectory 25 that extends dorsally and fits between trajectories 23, 24, and a lateral side trajectory 26 that extends dorsally and toward the radial styloid screw trajectory 21. The screw trajectories are optimized to ensure that when screws 12 are inserted through openings 16, the screws 12 capture the best possible bone stock, avoid joint penetration, and provide the most stable fixation for the healing of fractures in the wrist area.

One or more guide wires or K-wires may be supplied through the K-wire holes 20 to assist with preliminary placement of the plate 100. The guide wire or K-wire holes 20 offer additional points of fixation for the plate 100. Driving K-wires through the appropriate holes 20 in the plate 100 allows the plate 100 to be held on the bone while adjacent bone screws 12 can be inserted through the polyaxial holes 16. The proximal shaft 110 may include a proximal-most K-wire hole 20 aligned with the shaft central axis and a pair of K-wire holes 20 beneath and on opposite sides of the graft window 50. The distal head 112 of the plate 100 may include two to five distal K-wire holes 20 that are angled to a desired trajectory. The trajectories of these holes 20 may follow in parallel the screw trajectory of the nearest screw 12, providing direction during insertion of distal locking screws 12. The distal K-wire holes 20 may be tightly toleranced to ensure accurate trajectories.

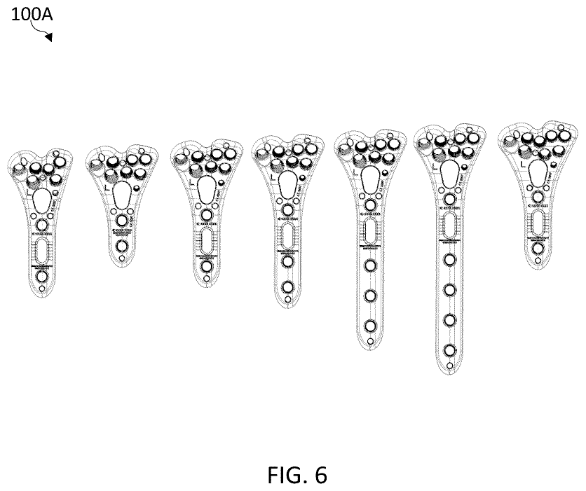

Turning now to FIG. 6, a collection of double row monoaxial volar plate styles 100A is shown. The double row monoaxial volar plates 100A are similar to the double row polyaxial plates 100 except the polyaxial locking holes 16 are replaced with monoaxial holes 18. The monoaxial plates 100A may accomplish the same goals as the distal radius double row volar plates, but with a thinner profile and more fixation options than the polyaxial counterpart. The monoaxial holes 18 are configured to engage a head portion of a fixed angle fastener 12, which may be locked to the plate 10 only in one designated direction. The monoaxial holes 18 may include locking holes with one or more threads extending between the top and bottom surface 106, 108 and/or stacked non-locking holes with non-threaded and threaded portions (e.g., shown in FIG. 11).

The monoaxial holes 18 may include fully threaded hole options as well as stacked holes for non-locking screws 12. The distal head 112 may define a double row of distal monoaxial holes 18. The double row of distal monoaxial holes 18 may be located on the ulnar-side and radial-side lobes 130, 132. In some embodiments, the distal-most row of monoaxial holes 18 may include four or five monoaxial holes 18. The second row of monoaxial holes 18 may be seated beneath the first row. The second row of distal monoaxial holes 18 may include two or three monoaxial holes 18.

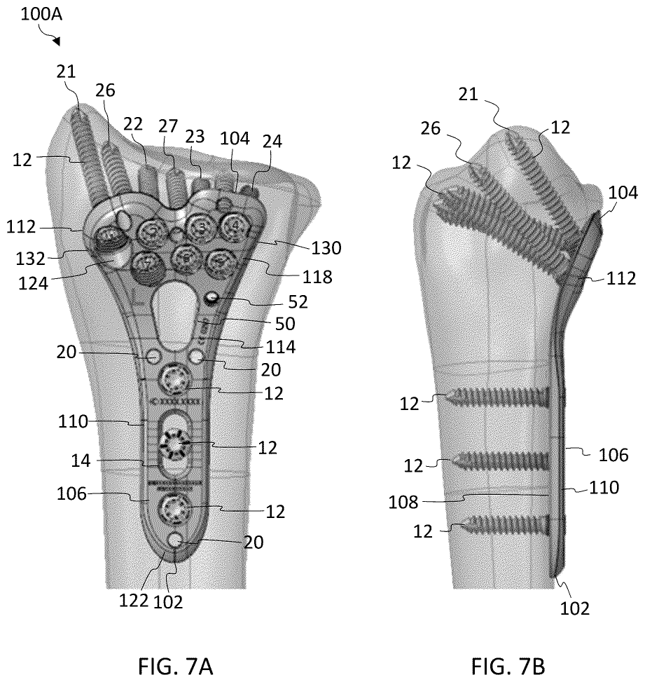

As shown in FIGS. 7A-7B, the optimized screw trajectories 21, 22, 23, 24 of the distal-most row of screws 12 may be aligned so that their nominal trajectories follow the articular surfaces of both the radiocarpal joint 21, 22, 23 and the distal radio-ulnar joint (DRUJ) 24. The radial-side lobe 130 includes screw trajectories 21, 22 and the ulnar side lobe 132 includes screw trajectories 23, 24. This allows the screws 12 to buttress and support the articular surfaces during fracture reconstruction. The radial styloid screw trajectory 21 provides the severe angle necessary to reach the very tip of the radial styloid. The area around the radial styloid screw hole 18 may include a smooth cut out 124 to accommodate the extreme angle of the radial styloid screw hole 18.

The second row of screw trajectories 25, 26, 27 may fit between the distal row of screws 12, supporting the articular surface by interdigitation. The ulnar-side lobe 130 includes screw trajectories 25, 27 and the radial-side lobe 132 includes screw trajectory 26. The ulnar side screw trajectories 25, 27 may interdigitate with the distal ulnar side screws 23, 24 and provide buttressing to the dorsal surface of the radius. The radial side trajectory 26 may extend dorsally and toward the radial styloid screw trajectory 21.

Similar to double plates 100, the double row monoaxial volar plates 100A may include two struts of material running up both the radial and intermediate columns of the bone, with a window 50 in between the two struts. The proximal shaft 110 may define a series of proximal monoaxial holes 18 aligned with the central axis of the implant 100A. When present, the sliding slot 14 may be aligned with and located between opposite monoaxial holes 18. Depending on the length of the plate 100A, the plate 100A may define between two and five monoaxial holes 18. For longer plates 100A, the additional monoaxial holes 18 may be located proximal to the sliding slot 14.

As best seen in FIG. 11, the monoaxial holes 18 include stacked holes with an upper unthreaded region 18A and a lower threaded region 18B aligned along the hole axis. The upper non-threaded region 18A may have a conical or spherical recess configured to receive the head of the fastener 12. The lower threaded region 18B may have a tapered diameter smaller than the upper non-threaded region 18A with one or more threads configured to engage the head of the fastener 12. The stacked monoaxial holes 18 (e.g., 2.5 mm monoaxial stacked non-locking hole) may be located along the shaft 110 of the plate 100A. The stacked holes 18 may be configured to accommodate locking or non-locking screws 12 in the same hole 18. This gives surgeons the option to use locking or non-locking screws 12 based on their preference. The monoaxial screw holes 18 may accept locking screws 12 at nominal angle trajectories to simplify the plate geometry.

The double row monoaxial volar plates 100A may include positioning slot 14 and K-wire holes 20. The positioning slot 14 may provide for proximal-distal (P-D) and/or medial-lateral (M-L) adjustment of plate 100A during provisional placement, such that the plate 100A may be optimally centered along the shaft of the radius prior to locking screw insertion. In one embodiment, three angled distal K-wire holes 20 may be sized and dimensioned to ensure accurate trajectories. The trajectories of the K-wire holes 20 may follow in parallel the radial styloid hole trajectory 21, the second row middle screw hole trajectory 27, and the distal ulnar screw hole trajectory 24, in order to facilitate proper placement of the plate 100A and to keep screws 12 out of the joint surface. A pair of proximal K-wire hole 20 may be provided in the proximal shaft 110 below the graft window 50. A proximal-most K-wire hole 20 may be located at the proximal end 102 of the plate 100A.

In this embodiment, the plate 100A may also include a targeting guide attachment hole 52, which is able to mate with a corresponding targeting guide, such as targeting guides 670, 700. The attachment hole 52 may define a female thread configured to receive a corresponding male thread on the targeting instrument. The attachment hole 52 may be located adjacent to the graft window 50, if present, and beneath the screw hole 18 for the second row ulnar side screw trajectory 25. It will be appreciated that the attachment hole 52 may be located at any suitable location, which does not interfere with the screw trajectories. It will also be appreciated that another attachment interface may be used to attach a suitable instrument to the plate 100A.

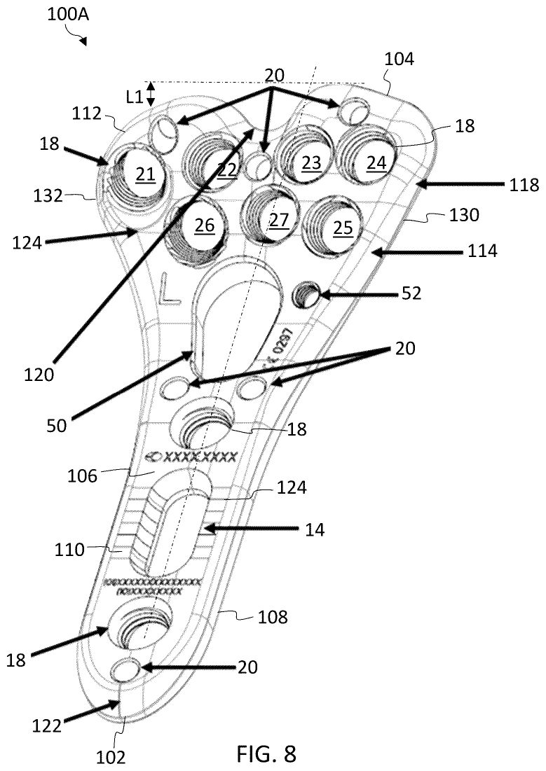

As best seen in FIG. 8, the edges of plate 100A may include a variable chamfered surface 118 on the distal end 112 of the plate 100A, which helps to ensure minimal tendon disruption by maintaining a low plate profile over the relevant tendon sites. The distal head 112 may define a flexor pollicus longus (FPL) tendon groove 120 configured to provide a smooth transition between bone and plate 100A for the flexor pollicis longus tendon to run over. The tendon groove 120 may include a concave recess in the distal edge 114 of the plate 100A forming two rounded or truncated lobes 130, 132 on either side of the shallow groove 120. In this embodiment, the ulnar-side lobe 130 is less rounded than the radial-side lobe 132 with a more angular shape. The ulnar-side lobe 130 may have a greater length L1, thereby causing ulnar-side lobe 130 to be longer or taller than the radial side lobe 132. The proximal end 102 of the shaft 110 may define tapered tip 122 configured for maintaining the low profile at the proximal end 120 of the plate 100A.

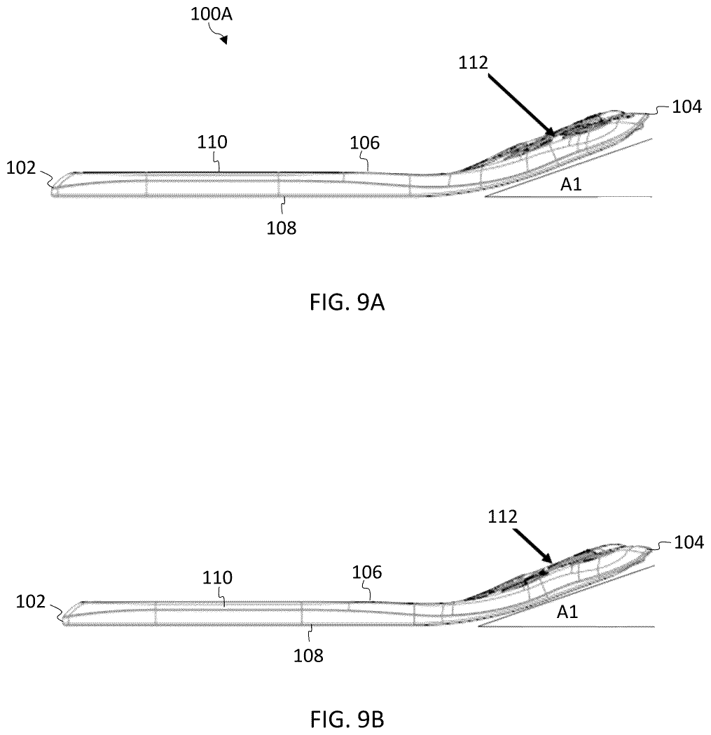

As shown in FIGS. 9A-9B, the anatomic contour along the bottom surface 108 of the plate 100A may follow the best approximation of average distal radius anatomy. As best seen in FIG. 9A, the volar monoaxial plates 100A may have a distal angulation A1 of 24° in order to have the plate 100A sit lower on the radius and not infringe on the Watershed line. The distal plate thickness may be optimized, for example, such that the plate 100A is held at 2 mm along the shaft and tapers to a thickness of 2.60 mm along the radial distal end 112 of the plate 100A, which allows for the severe angle of the radial styloid screw trajectory 21, and 1.80 mm across the ulnar-most distal hole 24 for strength. On the ulnar side 130, the plate thickness may taper to 1.30 mm across the distal-most K-wire hole 20, reducing the amount material closer to the Watershed line. As shown in FIG. 9B, monoaxial narrow implants may have less distal angulation A1) (22° than their standard and wide counterparts) (24° due to the population of smaller patients having radii that do not have as severe of a volar tilt as larger patients may have. This smaller distal angulation A1 in monoaxial narrow plates may prevent surgeons from having to pre- and intra-operatively bend plates to fit smaller patients.

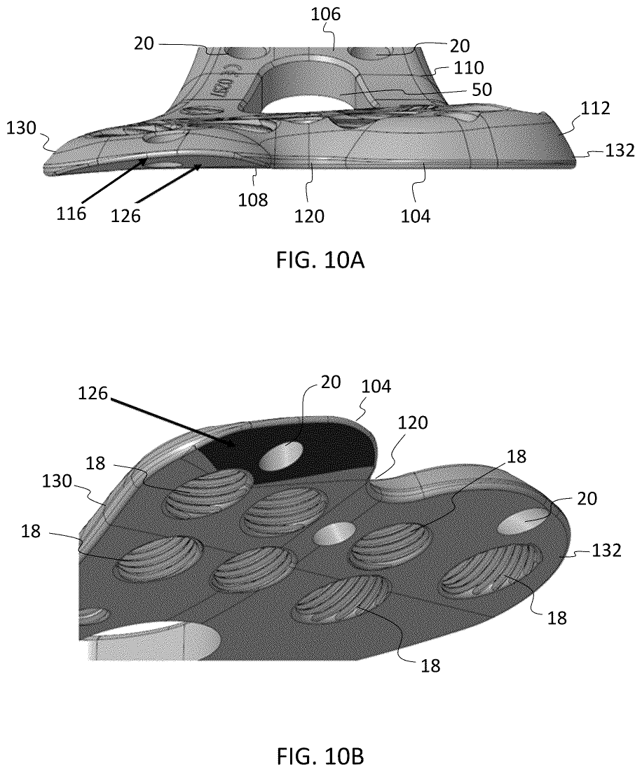

As shown in FIG. 10A, the distal edge contour is a curved profile configured to facilitate best fit of the contour on the ulnar side 130 of the radius. For example, the curved ulnar surface 116 may have a radius of 30 mm to mimic the curvature of the distal radius. As best seen in FIG. 10B, a distal ulnar undercut 126 may be provided along the bottom surface 108 at the distal-most end 104 of the plate 100A. The ulnar undercut 126 may promote the best fit on the ulnar side of the radius. The undercut 126 may provide the surgeon with extra space to slide the plate 100A distally towards the Watershed line while still facilitating proper fit.

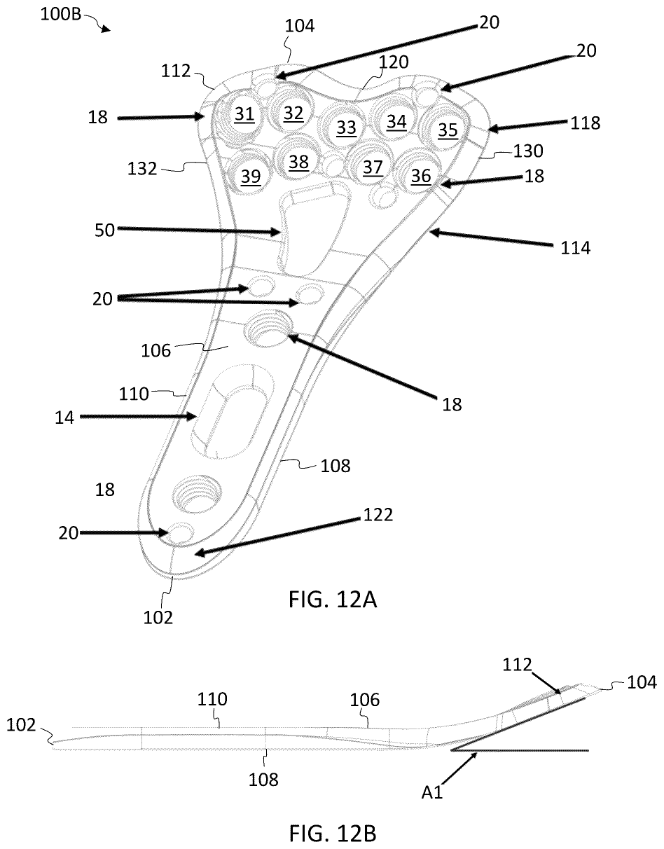

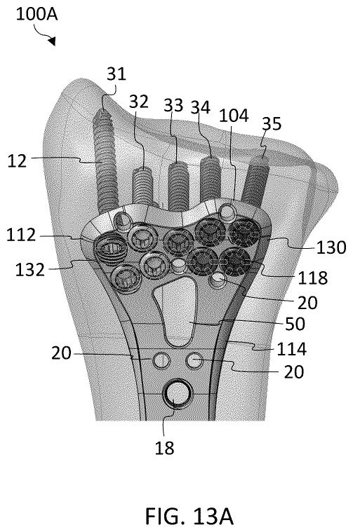

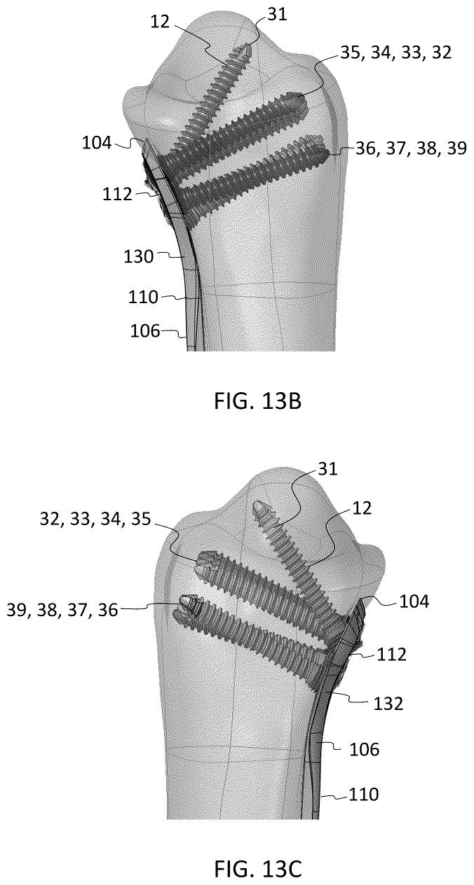

Turning now to FIGS. 12A-13C, a double row monoaxial volar plate 100B is shown according to another embodiment. The double row monoaxial volar plates 100B includes all of the same features as the double row monoaxial volar plates 100A with monoaxial locking holes 18 that are fully threaded between the top and bottom surfaces 106, 108.

As shown in FIG. 12A, plate 100B includes similar feature to plates 100, 100A. The variable chamfered surface 118 may be provided on the distal end 104 of the plate 100B to help to ensure minimal tendon disruption (specifically of the flexor pollicus longus and flexor carpi radialis) by maintaining a low plate profile over the relevant tendon sites. The plate 100B includes a single positioning slot 14 and K-wire holes 20. The positioning slot 14 allows for proximal-distal (P-D) and/or medial-lateral (M-L) adjustment of the plate 100B, such that the plate 100B may be optimally centered along the shaft of the bone prior to locking screw insertion. Four angled distal K-wire 20 holes may be provided in the distal head 112 to provide trajectories which follow the radiocarpal joint and provide direction during insertion of distal locking screws 12. A pair of midshaft K-wire holes 20 may be located beneath the bone graft window 50. A proximal-most K-wire hole 20 may be located near the tapered tip 122 of the proximal shaft 110.

The plate 100B includes a plurality of monoaxial locking holes 18 (e.g., 2.5 mm monoaxial locking holes). The monoaxial locking holes 18 are configured to accept locking fasteners 12 that, once inserted, lock into place at a single, predetermined angle relative to the plate 100B. The monoaxial locking holes 18 define internally threaded holes that are fully threaded between the top and bottom surfaces 106, 108, which engage matching threads on the head of the locking screw 12. When coupled, the locking screw 12 threads into the hole 18 creating a fixed angle construct, thereby preventing screw toggling. The distal monoaxial locking holes 18 in the distal head 112 may include two rows of holes. In this embodiment, nine distal locking holes 18 are provided in the distal head 112 of the plate 100B with the hole axes nominally aimed at the distal radius. Each hole axis may be nominally aimed at a different location such that the screws 12 follow individual trajectories that do not overlap. The proximal monoaxial locking holes 18 are located in the proximal shaft 110. For example, a pair of proximal monoaxial locking holes 18 may be located on either side of the positioning slot 14 to secure the plate 100B to the shaft of the bone.

As best seen in FIG. 12B, the plate 100B may have a pre-contoured geometry to fit the patient anatomy. The plate 100B may have a contour optimized on the distal head 112 to engage the distal radius. The anatomic contour along the bottom surface 108 of the plate 100B follows the best approximation of average distal radius anatomy. The distal head 112 of the monoaxial double row volar plates 100B may have a distal angulation A1 of 22° in order to have the plate 100B sit lower on the radius and not infringe on the Watershed line. The distal head 112 may have an optimized distal plate thickness to minimize interference and allow for reduced thickness near the Watershed line. For example, the plate 100B may be held at a thickness of 2 mm along the shaft 110, which tapers to a smaller thickness of 1.70 mm along the distal end 104 of the radial side 132 of the plate 100B and 1.25 mm along the distal end 104 of the ulnar side 130 of the plate 100B. In this manner, the distal head 112 has a thickness thinner than the proximal shaft 110, and the ulnar-side lobe 130 has a thickness thinner than the radial-side lobe 132.

As best seen in FIGS. 13A-13C, the screw trajectories of the distal clusters of screws 12 may be optimized so that their nominal trajectories follow the articular surfaces of both the radiocarpal joint 31, 32, 33, 34 and the distal radio-ulnar joint (DRUJ) 35. This allows the screws 12 to buttress and support the articular surfaces during fracture reconstruction. In this embodiment, up to nine locking fasteners 12 may be provided through the distal locking holes 18 in the distal head 112 of the plate 100B. Each hole axis may be nominally aimed at a different location such that the screws 12 follow individual trajectories that do not overlap. The radial styloid screw trajectory 31 provides the severe angle necessary to reach the very tip of the radial styloid. The second row screw trajectories 36, 37, 38, 39 may target the distal radius beneath the first row screws 32, 33, 34, 35 to secure the bone.

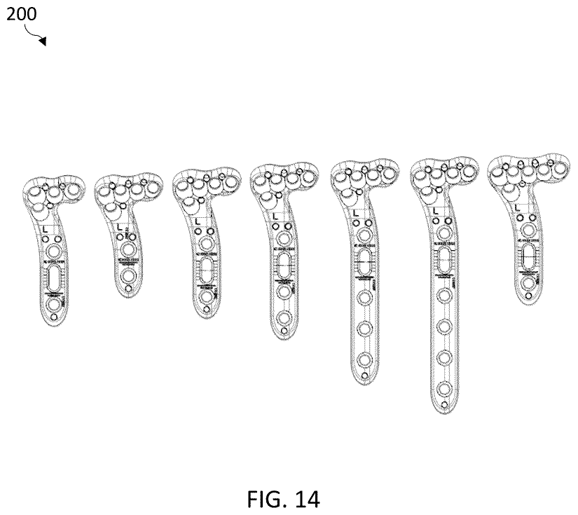

Turning now to FIGS. 14-16B, a collection of single row polyaxial volar plate styles 200 is shown. Single row polyaxial volar plates 200 are similar to double row polyaxial plates 100 except the proximal shaft 210 is a single strut without a graft window and the distal head 212 extends transversely from the shaft 210, terminating at a free end 230 on the ulnar side, and includes only a single row of polyaxial holes 16.

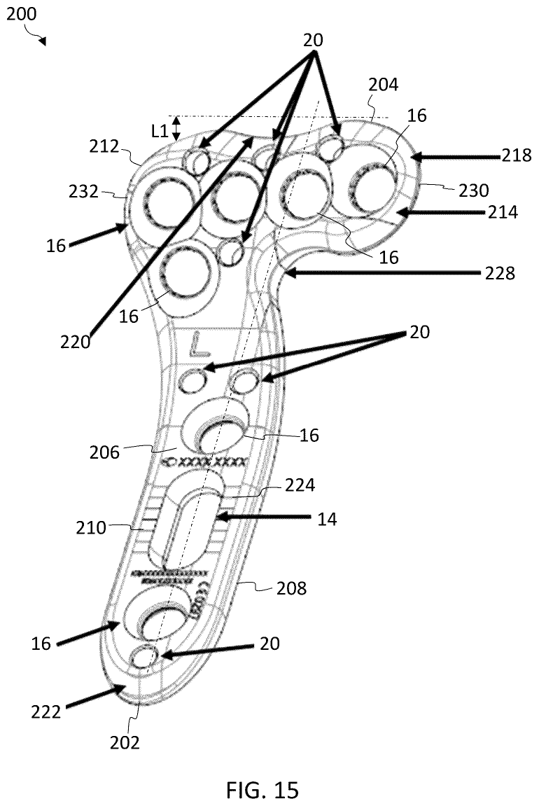

As shown in FIG. 15, the single row polyaxial volar plate 200 extends along central longitudinal axis from proximal end 202 configured to engage the radial shaft of the radius to distal end 204 configured to engage the distal radius. The plate 200 includes top surface 206 and bottom surface 208 with openings 14, 16, 20 extending therethrough. The plate 200 may include proximal shaft portion 210 and distal head 212 extending transversely therefrom. In this embodiment, the proximal shaft 210 is a single strut for visualization of the fracture line. The proximal shaft 210 curves and contours outward to transition to a radial side 232 of the distal head 212. The proximal shaft 210 may bend or curve into distal head 212 with a neck or transition region 228 that extends at an angle relative to the proximal shaft 210 and the distal head 212. The other end of the distal head 212 is a free end 230 on the ulnar side. A single row of distal polyaxial openings 16 may be provided through the distal head 212. An additional distal polyaxial opening 16 may be placed beneath the radial styloid opening to buttress and support that screw 12.

Similar to plates 100, the plate 200 may have a pre-contoured geometry and edges 214, which may be chamfered, for example, with a variable chamfered surface 218 to minimize tendon disruption. The distal end 204 may define a flexor pollicis longus (FPL) tendon groove 220, which defines two lobes 230, 232. The two rounded lobes 230, 232 may be non-symmetrical with one lobe 230, 232 being longer, wider, thicker/thinner and/or differently shaped than the other 230, 232. For example, the ulnar-side lobe 230 may have a greater length L1 and is longer than the radial side lobe 232. The tendon groove 220 is configured to provide a smooth transition between bone and plate 200 for the flexor pollicis longus tendon to run over to prevent tendon irritation and ruptures. The proximal end 202 of the shaft 210 may define a tapered tip 222, thereby maintaining a low profile at the proximal end 120 of the plate 200.

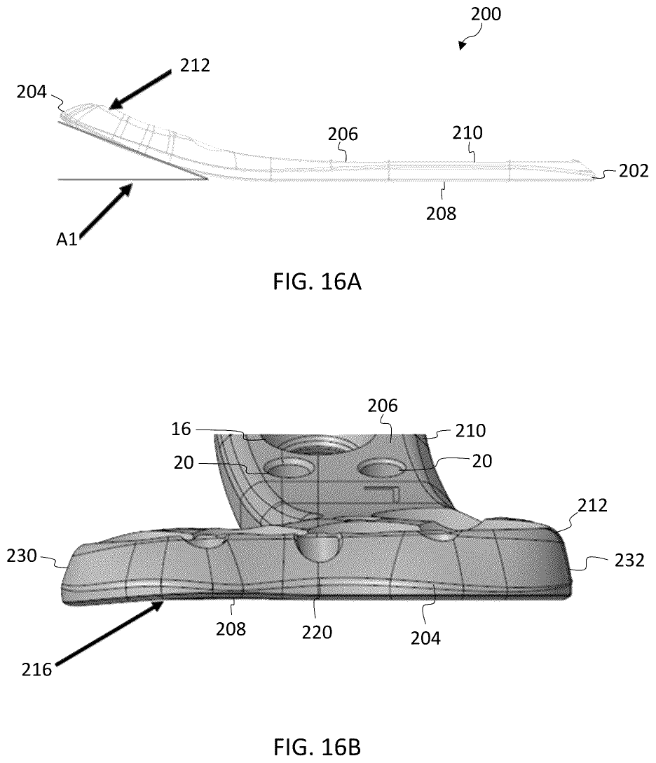

As best seen in FIG. 16A, the anatomic contour along the bottom surface 208 of the plate 200 may follow the best approximation of average distal radius anatomy. The single row polyaxial volar plates 200 may have a distal angulation A1, for example, of 24° in order have the plate 200 sit lower on the radius and not infringe on the Watershed line. In one embodiment, the thickness of the plate 200 may be held at 2 mm along the proximal shaft 210 and tapers to an increased thickness of 2.75 mm along the distal head 212 of the plate 200. As best seen in FIG. 16B, a curved ulnar surface 216 may be located on the bottom surface 108 to promote best fit of the distal head 212 with the distal radius. The curved ulnar surface 216 may extend to the free end 230 on the ulnar side 230 of the distal head 212.

Similar to plates 100, plates 200 include sliding slot 14 along the proximal shaft 210, K-wire holes 20, and polyaxial locking holes 16 along the distal head 212 and proximal shaft 210. The sliding slot 14 may provide for proximal-distal (P-D) adjustment as well as medial-lateral (M-L) adjustment of the plate 200 during provisional placement, thereby allowing for optimal positioning along the radial shaft of the bone prior to locking screw insertion. Four angled distal K-wire holes 20 may be located in the distal head 204 to ensure accurate trajectories. The trajectories of the K-wire holes 20 may follow the same screw trajectories of the nearest screws 12, providing direction during insertion of distal locking screws 12. In the same manner as the shallow contour double row plates 100, the screw trajectories of the distal row of screws 12 are configured to be aligned so that their nominal trajectories follow the articular surfaces of both the radiocarpal joint and the distal radio-ulnar joint. This allows the screws 12 to buttress and support the articular surfaces during fracture reconstruction. The radial styloid screw opening 16 provides the severe angle necessary to reach the very tip of the radial styloid.

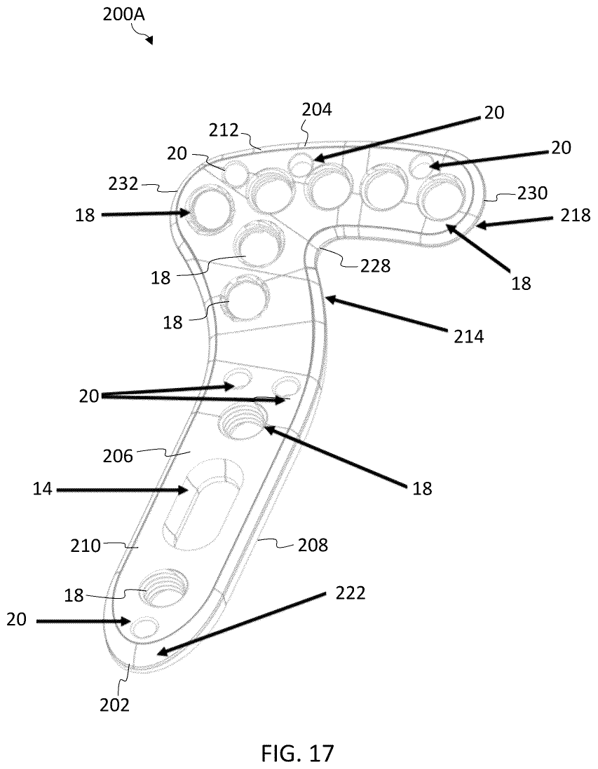

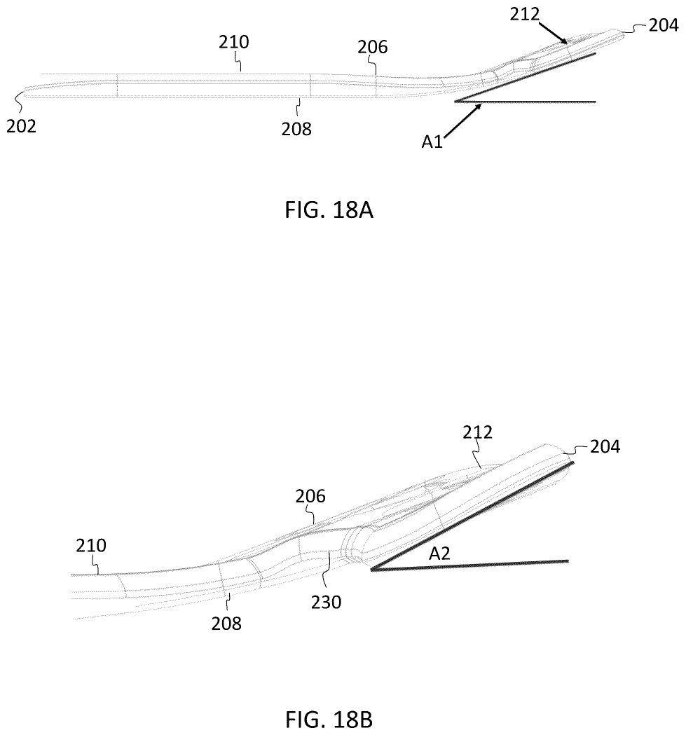

Turning now to FIGS. 17-18B, a single row monoaxial volar plate 200A is shown according to one embodiment. Single row monoaxial volar plate 200A is similar to single row polyaxial volar plates 200 except the polyaxial holes 16 are replaced with monoaxial holes 18 that are fully threaded between the top and bottom surfaces 206, 208.

Similar to the monoaxial double row plate 100B, the screw trajectories of the distal row of screws 12 have been optimized for strategic placement and angulation configured to align with the anatomical structures and bone quality of the distal radius. For example, seven distal monoaxial locking holes 18 (e.g., 2.5 mm locking holes) may be provided through the plate 200A. Five monoaxial locking holes 18 may be aligned through the distal head 212 and additional monoaxial holes 18 may be located beneath the distal row of holes 18. The screw trajectories may be aligned so that their nominal trajectories follow the articular surfaces of both the radiocarpal joint and the distal radio-ulnar joint. This allows the screws 12 to buttress and support the articular surfaces during fracture reconstruction. The radial styloid screw 12 provides the severe angle necessary to reach the very tip of the radial styloid. The two additional monoaxial holes 18 may be located beneath the second hole in the distal row to interdigitate between the distal row of screws 12 or otherwise buttress and support the articular surface.

A pair of monoaxial holes 18 may be provided through the proximal shaft 210 to secure the plate 200A to the radial shaft. The proximal shaft 210 may further include sliding slot 14 for proximal-distal (P-D) as well as medial-lateral (M-L) adjustment of the plate 200A during provisional placement. Up to three angled K-wire holes 20 may be positioned in the distal head 212 of the plate 200A. The distal K-wire holes 20 may be tightly toleranced to ensure accurate trajectories to follow the radiocarpal joint and provide direction during insertion of distal locking screws. The proximal shaft 210 may also include two straight midshaft K-wire holes 20 and a straight proximal K-wire hole 20 for provisional placement of plate 200A.

As best seen in FIG. 17, the variable chamfered surface 218 may extend along the distal head 212 to avoid the Watershed line of the volar distal radius, which may help to ensure minimal tendon disruption by maintaining a low plate profile over the relevant tendon sites. Similar to the monoaxial double row plate 100B, the thickness of the monoaxial single row plate 200A may be optimized to provide for a reduced thickness near the Watershed line. For example, the plate thickness may be held at 2 mm along the proximal shaft 210, which tapers to a decreased thickness of 1.75 mm along the distal end 204 of the radial side 232 of the plate and 1.10 mm along the distal end 204 of the ulnar side 230 of the plate 200A.

As best seen in FIG. 18A, similar to the monoaxial double row plate 100B, the anatomic contour along the bottom surface 208 of the plate 200A may follow the best approximation of average distal radius anatomy. The single row volar plates 200A may have a distal angulation A1, for example, of 22° in order have the plate 200A sit lower on the radius and not infringe on the Watershed line, thereby minimizing soft tissue and tendon irritation. As best seen in FIG. 18B, the single row volar plate 200A may have a dorsal tilt A2, for example, of 10° to facilitate a better fit on the ulnar side 230 of the radius. The ulnar side 230 of the plate 200A may be contoured for an improved plate fit.

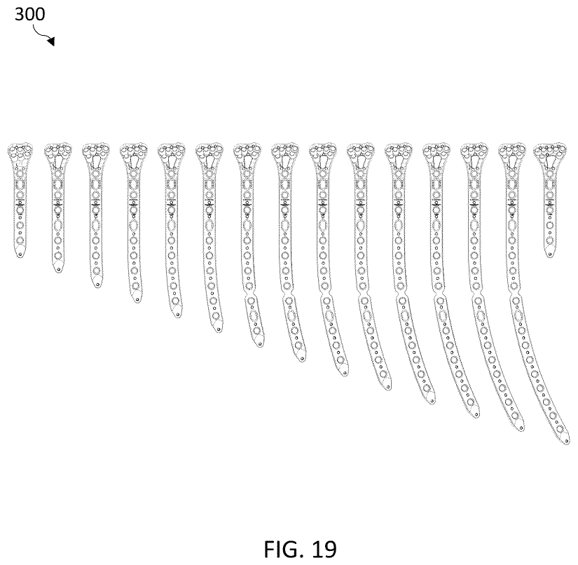

Turning now to FIGS. 19-21, a collection of diaphyseal-metaphysical plates 300 is shown. The diaphyseal-metaphyseal plates 300 are configured to sit on the volar face of the radius. The diaphyseal-metaphyseal plate 300 may be used for proximal volar fractures of the distal radius. The distal head 312, plate contour, and screw trajectories may be the same as the double row polyaxial volar plate 100, but the diaphyseal-metaphyseal plate 300 has a different proximal shaft 310 to accommodate the sagittal twist of the radius. The diaphyseal-metaphyseal plates 300 are configured to run the length of the radius to address volar and midshaft radius fractures with one plate. The diaphyseal-metaphyseal plates 300 allow surgeons to address midshaft and volar radius fractures with a single plate instead of two separate plates.

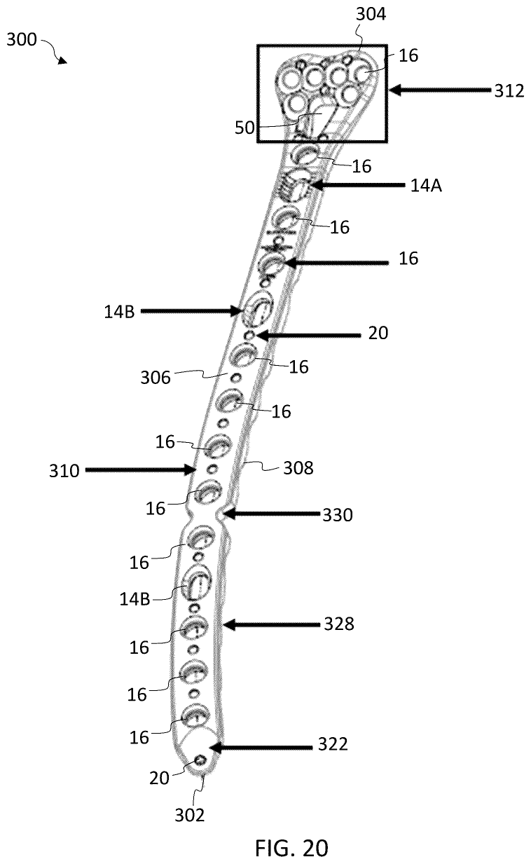

As best seen in FIG. 20, the diaphyseal-metaphyseal plate 300 has a body that extend along a central longitudinal axis from a first end or proximal end 302 configured to sit on the radial shaft to a second end or distal end 304 configured to sit on the distal end of the radius. The diaphyseal-metaphyseal plate 300 includes a top surface 306 and an opposite, bottom surface 308 configured to contact adjacent bone. The plates 300 are configured in both left and right designs, in a mirrored configuration, in order to address the anatomy of both the left and right arms of the patient.

The plate 300 includes a proximal shaft 310 and an enlarged distal head 312. The attributes of the distal head 312 are the same as head 112 for the double row polyaxial volar plate 100. The proximal shaft 310 may have an elongated longitudinal body, having a length greater than its width. The proximal shaft 310 may have a pre-contoured geometry to follow the bend of the radius bone. The proximal shaft 310 may be straight or curved, with larger lengths having a greater degree of curvature to account for the natural curvature and rotational shape of the radius bone when viewed from the sagittal plane. The proximal shaft 310 of the longer plates 300 may include a sagittal twist of up to 10° to accommodate the sagittal twist of the radius. For example, plates 300 longer than ten holes 15 may have 10° of sagittal twist. The proximal end 302 of the proximal shaft 310 may have a tapered tip 322 configured for maintaining the low profile at the proximal end 320 of the plate 300. The tapered tip 322 narrows and becomes thinner towards the proximal end 320, which is situated toward the elbow, in order to reduce the prominence of the plate 300 under the skin and to minimize irritation of overlying soft tissues, such as tendons or muscles.

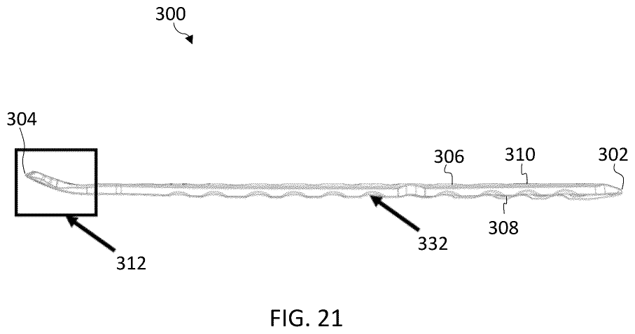

The proximal shaft 310 may include scallops or side cuts 330 to promote bending. For example, the proximal shaft 310 may include a pair of side cuts 330 partially down the shaft 310 for ease of medial/lateral plate bending. As best seen in FIG. 21, the proximal shaft 310 may further include scalloped undercuts 332 to minimize de-vitalization of the periosteum. The scalloped undercuts 332 may include recesses having partially curved valleys cut around a periphery of the bottom surface 108 of the plate 300. The scalloped undercuts 332 may reduce contact between the plate 300 and the bone surface, thereby helping to preserve blood supply to the bone and prevent osteonecrosis.

The shaft thickness may be increased for strength and to accommodate larger locking and non-locking polyaxial holes 16. The thicker shaft 310 allows for adequate support in the radial shaft. In one embodiment, the proximal shaft 310 may have a greater thickness, for example, a thickness of 3.3 mm to accommodate larger polyaxial locking holes 16. The proximal shaft 310 may include a series of polyaxial holes 16 (e.g., 3.5 mm polyaxial holes) aligned along the straight or curved longitudinal axis of the plate 300. The shaft holes 16 may accommodate locking and non-locking screws 12. For example, the shaft holes 16 may include polyaxial stacked holes having an upper non-threaded portion and a lower threaded portion (e.g., as shown in FIG. 11). The polyaxial holes 16 may provide for variable angle screw insertion, for example, with up to 40 degrees of cone of angulation. Depending on the length of the plate 300, the proximal shaft 310 may include up to sixteen polyaxial fastener openings 16.

The proximal shaft 310 may define one or more slots 14 for plate positioning and/or dynamic compression of the bone. The proximal shaft 310 may include a positioning slot 14A (e.g., a 3.5 mm positioning slot) toward the distal end 304 of the proximal shaft 310. The positioning slot 14A may accommodate non-locking screws 12 to provide for provisional placement of the plate 300. The proximal shaft 310 may include one or more optional dynamic compression slots 14B (e.g., a 3.5 mm compression slot) at a location proximal to the positioning slot 14A. For example, two polyaxial locking holes 16 may be positioned through the proximal shaft 110 between the positioning slot 14A and the compression slot 14B. For longer plate constructs, a second compression slot 14B may be located proximate to the side cuts 330. The dynamic compression slots 14B may allow for bi-directional dynamic compression through static insertion of a non-locking screws 12 into the bone and/or compression along the bone through eccentric insertion of the non-locking screw 12.

The proximal shaft 310 defines a plurality of guide wire or K-wire holes 20 for provisional placement and/or screw insertion guidance. The shaft K-wire holes 20 may be centered on the central longitudinal axis, straight or curved, of the proximal shaft 310. In one embodiment, the K-wire holes 20 are located every other hole 16 except between the side cuts 330. A proximal-most K-wire hole 20 may be located through the tapered tip 322. The shaft configuration allows for an easy transition between fixation in the head of the distal radius and the shaft of the radius.

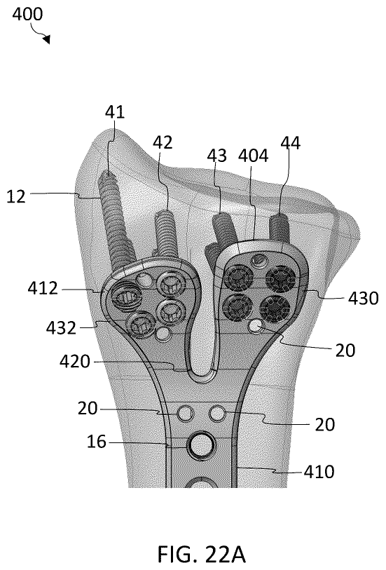

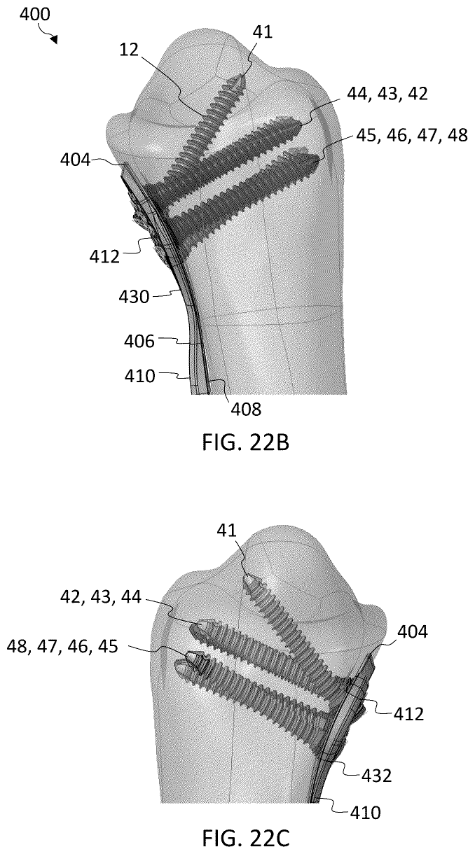

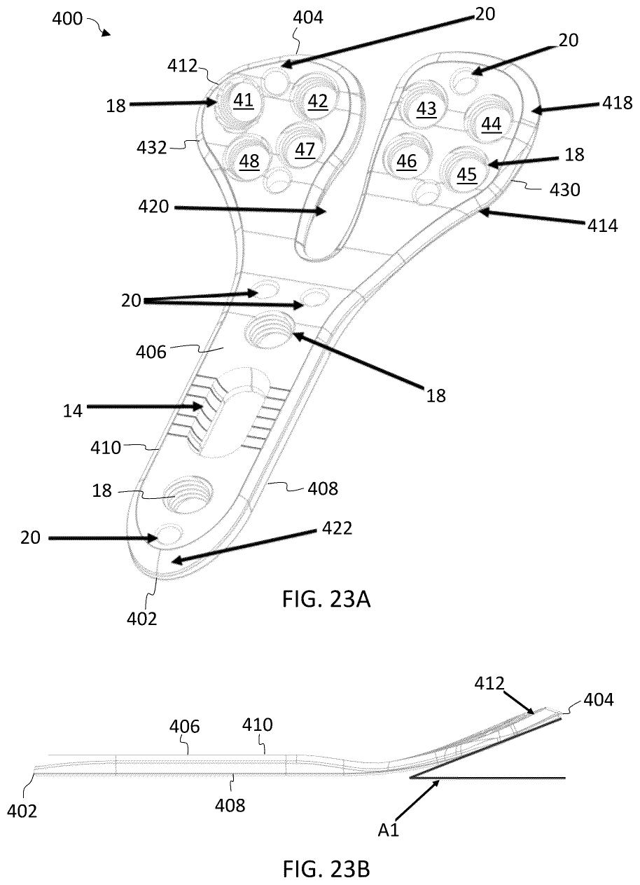

Turning now to FIGS. 22A-23B, a monoaxial flexor pollicis longus (FPL) volar plate 400 is shown according to one embodiment. The monoaxial FPL volar plate 400 is similar to the double row monoaxial volar plate 100B with a more pronounced flexor pollicis longus tendon groove 420, which is configured to receive a portion of the tendon to prevent tendon irritation and ruptures.

FIGS. 22A-22C depict the anatomy of the distal radius with the monoaxial FPL volar implant 400 contoured to sit on the volar aspect of the distal radius. The monoaxial FPL volar implant 400 is configured to be positioned against an outside face of the bone, for example, of the distal radius, and secured with fasteners 12. The monoaxial FPL volar implant 400 has a body that extends from a first end or proximal end 402 configured to sit on the radial shaft to a second end or distal end 404 configured to sit on the distal end of the radius. The plates 400 are configured in both left and right designs, in a mirrored configuration, in order to address the anatomy of both the left and right arms of the patient.

The distal head 412 is bifurcated into two pronounced distal heads or lobes 430, 432 by the tendon groove 420. The ulnar-side lobe 430 is configured to sit on the ulnar side of the distal radius and the radial-side lobe 432 is configured to sit on the radial styloid side of the distal radius. The ulnar-side lobe 430 may have a greater length L1 and is longer than the radial side lobe 432. The two rounded lobes 430, 432 may be non-symmetrical, for example, about the central axis, with one lobe 430, 432 being longer, wider, thicker/thinner and/or differently shaped than the other 430, 432. In this embodiment, the radial-side lobe 432 is more rounded than the ulnar-side lobe 430 where the ulnar-side lobe 430 is more angular or truncated than the radial-side lobe 432.

The tendon groove 420 may be generally centered or off-center on the plate 400 while providing a deep amorphous groove. The groove 420 may be an irregularly shaped channel or depression extending toward the proximal end 402 of the plate 400. The tendon groove 420 may extend at least one third of the length of the plate 400, which then transitions into the proximal shaft 410. The tendon groove 420 provides less material under where the FPL tendon runs along the distal radius, thereby minimizing tendon irritation. The two separate distal heads or lobes 430, 432 may be adjusted independent of each other intraoperatively, for example, to optimize placement against the bone and screw trajectories. Each lobe 430, 432 of the distal head 412 may include two rows of monoaxial holes 18. The monoaxial holes 18 are configured to receive fixed angle fasteners 12, which may be locked to the plate 400 only in one designated direction. The monoaxial holes 18 may include locking holes with one or more threads extending between the top and bottom surface 406, 408.

The screw trajectories of the distal clusters of screws 12 may be aligned so that their nominal trajectories follow the articular surfaces of both the radio-carpal joint 41, 42, 43 and the distal radio-ulnar joint 44. The radial-side lobe 432 includes first row trajectories 41, 42 and the ulnar-side lobe 430 includes first row trajectories 43, 44. This allows the screws 12 to buttress and support the articular surfaces during fracture reconstruction. The radial styloid screw trajectory 41 provides the severe angle necessary to reach the very tip of the radial styloid. The second row of distal screws 12 may have trajectories 45, 46, 47, 48 that sit below the distal-most row of screws 12. The ulnar-side lobe 430 includes trajectories 45, 46 and the radial-side lobe 432 includes trajectories 47, 48. The screw trajectories may be optimized to ensure that when screws 12 are inserted through openings 18, the screws 12 capture the best possible bone stock, avoid joint penetration, and provide the most stable fixation for the healing of fractures in the wrist area. Four angled distal K-wire holes 20 may be provided in the distal end 404 to ensure accurate trajectories to follow the radiocarpal joint and provide direction during insertion of distal locking screws 12.

The proximal shaft 410 may include the same features as double row monoaxial volar plate 100B including tapered tip 422, monoaxial shaft holes 18, positioning slot 14, and proximal K-wire holes 20. As shown in FIG. 23B, the anatomic contour along the bottom surface 408 of the plate 400 follows the best approximation of average distal radius anatomy. The FPL volar plates 400 may have a distal angulation A1, for example, of 25° in order have the plate 400 sit lower on the radius and not infringe on the Watershed line. A significant variable chamfered surface 418 on the distal end 404 of the plate 400 helps to ensure minimal tendon disruption by maintaining a low plate profile over the relevant tendon sites. The thickness of the FPL plate 400 may be held at 2 mm along the proximal shaft 410 and tapers to a decreased thickness of 1.85 mm along the distal end 404 of the radial side 432 of the plate and 1.30 mm along the distal end 404 of the ulnar side 430 of the plate 400, which allows for reduced thickness near the Watershed line.

The distal radius volar plates described herein offer enhanced visualization of the fracture site and optimized screw trajectories. The two-axis sliding slot allows for provisional placement and centering of the plate on the bone. The volar plates are configured, for example, with thinner head thickness and optimized distal contour to minimize soft tissue irritation in the volar region. The tendon groove is provided to prevent tendon irritation and reduce the likelihood of tendon ruptures. The collection of plates include a comprehensive offering to treat a vast array of fracture patterns in the distal radius. The plates may be used for both definitive, permanent fixation, as well as temporary or supplemental fixation. The specific plate styles afford the ability to accommodate multiple fracture patterns and various anatomies and anatomic regions.

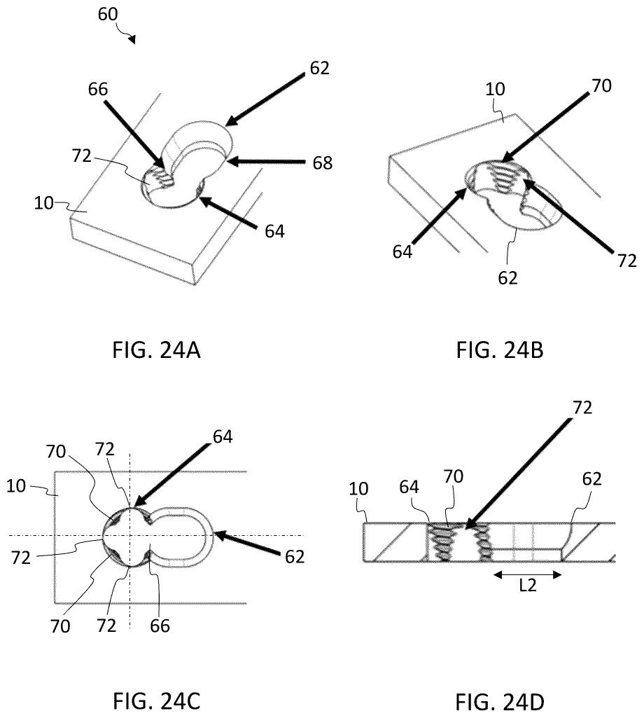

Turning now to FIGS. 24A-31C, any of the fastener slots 14 or holes 16, 18 described herein may be replaced with combined or merged holes 60, 60A configured for the interfragmental compression of fractures and fragments in many different anatomies. For plating of diaphyseal bone, many surgeons use a combination of both locking and non-locking screws that are able to dynamically compress bone, to connect the bone and plate and also create interfragmental compression while tightening the screws 12. Instead of separate openings, combined or merged holes 60, 60A may provide for compression of fracture fragments and locking functionality.

The combined holes 60, 60A offer the option to either lock the plate to bone or dynamically compress the fracture fragments in a single opening, so the surgeon does not have to drill eccentrically in a single locking hole to achieve the necessary compression. The combined or merged holes 60 may include two overlapping holes: a non-threaded hole 62 merged with a threaded hole 64. These holes 60, 60A are used for compressing fracture fragments together when screws 12 are tightened. Both holes 60, 60A have a non-threaded, non-locking side 62 and a partially threaded locking side 64. The dynamical compression is achieved through the ability to have options between locking and non-locking. The partial threads in the locking side 62 may be configured to create a polyaxial construct, thereby locking the fastener 12 with a screw angle of up to 15° in any direction.

FIGS. 24A-27B show a short combination opening or merged hole 60. As best seen in FIGS. 24A-24D, the short combination opening or merged hole 60 may include a hole formed of two holes: one non-threaded 62 and one threaded 64, joined by an overlap 66 to facilitate dynamic compression across the fracture line. The non-threaded portion 62 may be elongated a length L2 along the longitudinal axis of the plate 10. The inner surface of the non-threaded portion 62 may have a substantially smooth surface 68 such that the non-locking fastener 12 is able to freely pivot and/or slide along length L2 of the non-threaded portion 62. This provides for at least two directions of compressive force (e.g., along the longitudinal axis and perpendicular to the longitudinal axis of the plate 10).

The head portion of the non-locking fastener 12 may be substantially smooth around its outer surface. The head portion of the non-locking fastener 12 is sized and configured to engage with and be retained within the non-threaded portion 62 of the merged hole 60 (e.g., 2.5 mm hole). The non-threaded portion 62 may be configured to receive a fixed or variable angle fastener 12. In one embodiment, the non-threaded portion 62 may be generally conical or spherical in shape and/or tapered such that it is wider near the top surface of the plate 10 and narrower toward the bottom surface of the plate 10. In this embodiment, the non-threaded portion 62 is a smooth variable angle hole configured to receive the non-locking fastener 12 (e.g., 2.5 mm non-locking screw), thereby allowing movement of the fastener 12, for example, in a polyaxial fashion and/or along the length of the non-threaded portion 62, thereby providing dynamic compression of the bone.

The threaded portion 64 may be a cylindrical or circular hole along the longitudinal axis of the plate 10 and aligned with the non-threaded portion 62. The threaded portion 64 may define partial threads 66 or another textured surface, which facilitate locking with a locking fastener (e.g., a 2.5 mm locking screw). The threads 66 may be interrupted by material cutouts 72 that facilitate polyaxial movement of the screw 12. The material cutouts 72 may include concave recesses extending between the top and bottom surfaces of the plate 10.

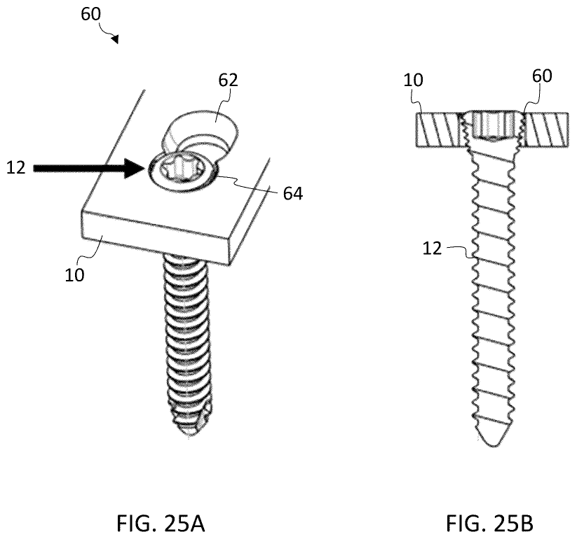

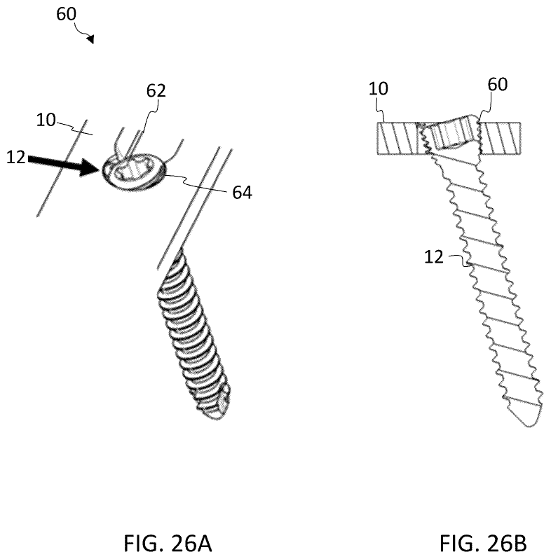

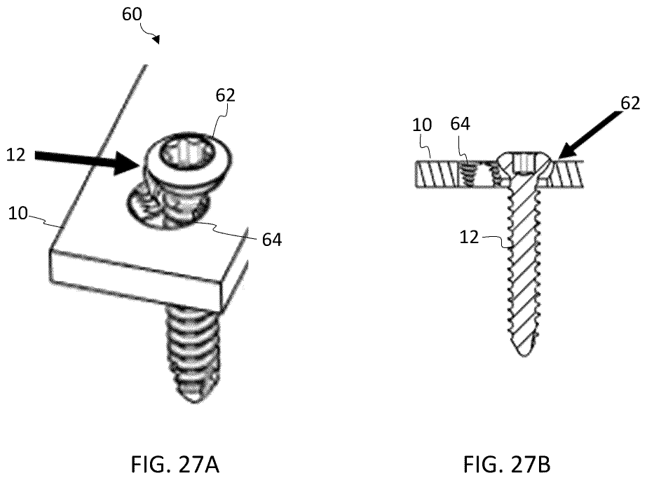

The material cutouts 72 and threaded sections 66 may alternate to form a clover-like shape. The material cutouts 72 may be located symmetrically about the threaded hole axis. For example, the material cutouts 72 may be centered on the x and y axes equidistant from the threaded hole axis. These sections of removed threads 72 allow the locking screw 12 to have a polyaxial trajectory. For example, the locking screw 12 may be angled up to 15° in any direction due to the clover-like shape of material cut out of the locking hole 64. FIGS. 25A-25B show one example of the locking screw 12 at a nominal trajectory through the polyaxial locking clover 64 of the merged hole 60. In this embodiment, the locking screw head male threads engage with the female threads in the merged hole 60 to create a locked construct. FIGS. 26A-26B show the locking screw 12 at a polyaxial trajectory. The polyaxial locking clover 64 may facilitate a polyaxial cone of angulation up to 15°, for example. FIGS. 27A-27B show the non-locking screw 12 at a nominal trajectory. The non-locking screw 12 may be inserted at nominal trajectory, parallel to the axis at the center of the non-locking hole 62.

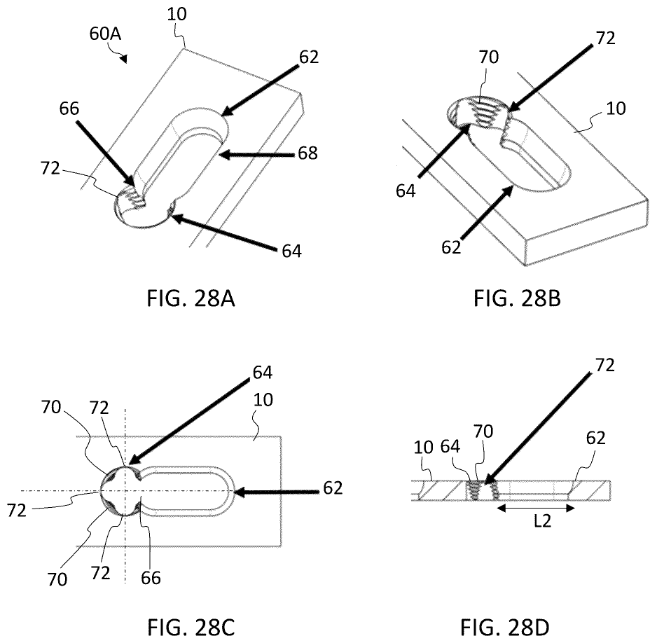

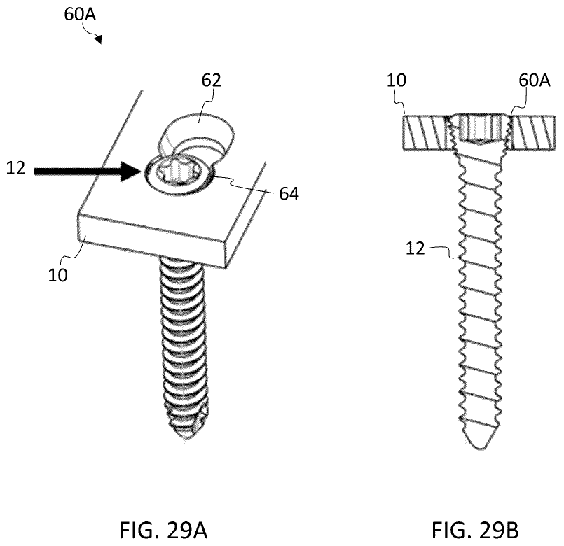

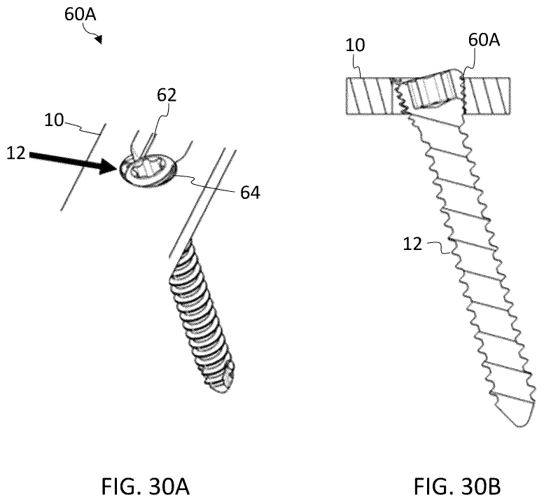

FIGS. 28A-31C show an oblong combination opening or merged hole 60A. The oblong combination opening 60A is the same as the short combination opening 60 except the non-locking hole 62 has a longer oblong slot for plate positioning. In this embodiment, the length L2 of the non-threaded portion 62 is greater than for the short hole 60. As shown in FIG. 29A-29B, in the same manner as merged opening 60, the locking screw 12 (e.g., 2.5 mm locking screw) may be provided at a nominal trajectory such that the head male threads engage with the female threads 70 in the merged hole 60A to create a locked construct. As best seen in FIG. 30A-30B, the locking screw 12 may also be angled 15° in any direction due to the clover-like shape of material cut out 72 of the locking hole 64. These sections of removed threads 70 allow the locking screw 12 to have a polyaxial trajectory.

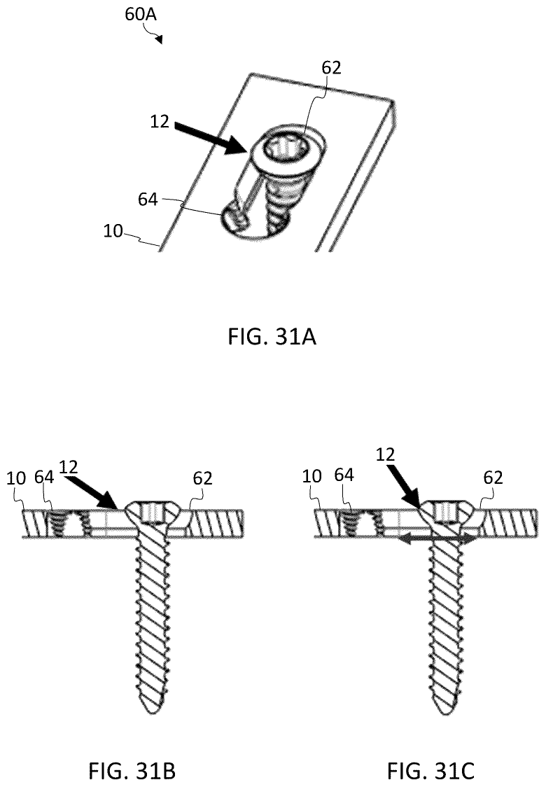

As shown in FIGS. 31A-31B, the non-locking screw 12 (e.g., 2.5 mm non-locking screw) may be inserted at a nominal trajectory, parallel to the center of the non-locking opening 62. The non-locking screw 12 may be inserted anywhere in the oblong hole 62. With further emphasis on FIG. 31C, the non-locking screw 12 may be inserted along the length L2 of the oblong hole 62, and the plate 10 may be moved proximally or distally along the bone while held in position on the bone by the non-locking screw head and shaft before final tightening of the screw 12 is performed. The oblong combination hole 60A is a longer version of the short combination hole 60, with the non-threaded overlapped hole 62 being longer to give the surgeons the ability to position the plate proximally or distally during intra-operative technique.

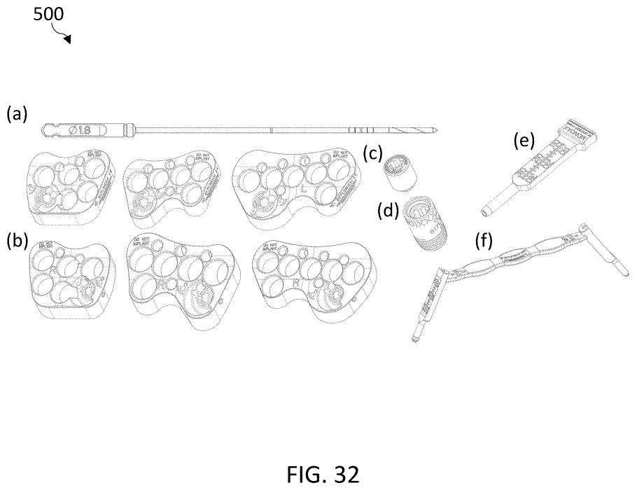

Turning now to FIGS. 32-50B, a number of different instruments are shown for installing the distal radius plates. As shown in FIG. 32, one example of a set of instruments 500 is shown including: (a) a calibrated drill bit; (b) a collection of polyaxial volar targeting guides; (c) a K-wire sleeve; (d) a monoaxial locking drill guide; (e) a measuring block for the monoaxial volar targeting guide; and (f) a calibrated polyaxial drill guide. The set 500 may also include diaphyseal metaphyseal plate trials, monoaxial targeting guides, and other instruments for performing the procedure. These instruments 500 may be provided in a number of variations in a surgical tray such that the surgeon may select the desired instrumentation during surgery.

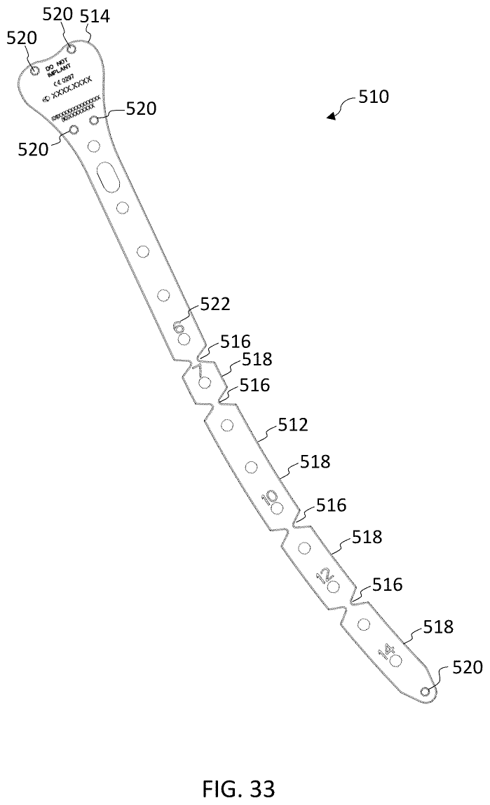

FIG. 33 shows one embodiment of a diaphyseal metaphyseal plate trial 510. The diaphyseal-metaphyseal plate trial 510 provides a system to anticipate the correct plate size that will be required for the surgery without dirtying an implant. The diaphyseal metaphyseal plate trial 510 has a geometry that corresponds to the diaphysical-metaphysical plates 300. The plate trial 510 has an elongated proximal shaft 512 and an enlarged distal head 514 configured to match the respective shaft and head of the diaphyseal-metaphysical plates 300.

The trial 510 may be shortened by breaking off tabs 518 along the proximal shaft 512. The proximal shaft 512 may include sizing scallops 516, which separate the shaft 512 into distinct tabs 518. The sizing scallops 516 may include V-shaped recesses cut into opposite sides of the trial 510. The sizing scallops 516 may be provided at the transition of each plate size to allow surgeons to decide which size plate will be needed for surgery. The material of the plate trial allows the tabs 518 to be twisted off and removed by hand to provide the desired length. The trial 510 may include one or more K-wire holes 520 to temporarily align the trial 510, shortened to the desired length, at the surgical site.

The trial 510 includes markings 522, such as numbering, that corresponds with the plate sizes. The trial 510 may include one or more markings 522, such as indicators, etchings, hole locations, numbers, etc. which indicate the number of holes remaining for sizing the corresponding plate 300. The markings 522 may include hole numbers on the plate trial 510 corresponding to the hole numbering of the diaphysical-metaphyseal plates 300. The markings 522 may also include circles or ovals indicating the anticipated locations of the holes and slots on the plates 300.

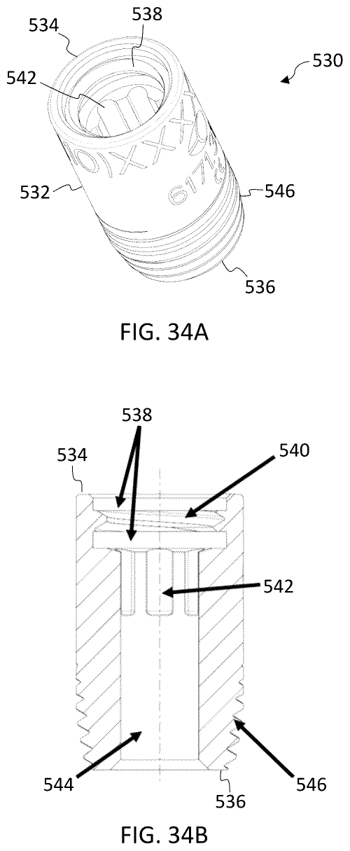

Turning now to FIGS. 34A-34B, a monoaxial locking drill guide 530 is shown according to one embodiment. The monoaxial locking drill guide 530 is configured to lock into the monoaxial holes 18 of the monoaxial distal radius plates 100A or other suitable plates to precisely position and guide the drilling of holes for the insertion of screw 12 into the plates 100A. The drill guide 530 may include a cylindrical body 532 extending from a proximal end 534 to a distal end 536 with a central cannulation. The drill guide 530 defines a counterbore 538, a threaded recess 540, a drive recess 542, and a drill cannulation 544 along the central tool axis. The threaded recess 540 (e.g., a 3 mm diameter 0.5 mm pitch thread) in the top of the drill guide 530 facilitates the connection of a threaded K-wire sleeve 550. The proximal counterbore 538 accepts a threaded K-wire sleeve 550. The counterbore 538 above and below the threaded recess 540 allows for seamless connection of the K-wire sleeve 550 to the drill guide 530. The drive recess 542 may include a six-point star-shaped pattern, hexalobular profile, torx profile (e.g., T8), or other suitable recess for engaging a driver instrument that facilitates the attachment and removal of the drill guide 530 to/from the monoaxial distal radius plates 100A. The inner diameter of the drill guide 530 is a drill cannulation 544 that provides a bearing surface to keep the drill on-axis. The bearing surface optimizes the tolerance stack and depth of counterbore 538. An exterior surface of the drill guide 530 includes a tapered thread 546 configured to lock into the monoaxial holes 18 in the distal head 112 of the monoaxial distal radius plates 100A. The drill guide 530 may be calibrated to obtain a screw measurement (e.g., from 16-24 mm) using the top of the locking drill guide 530.

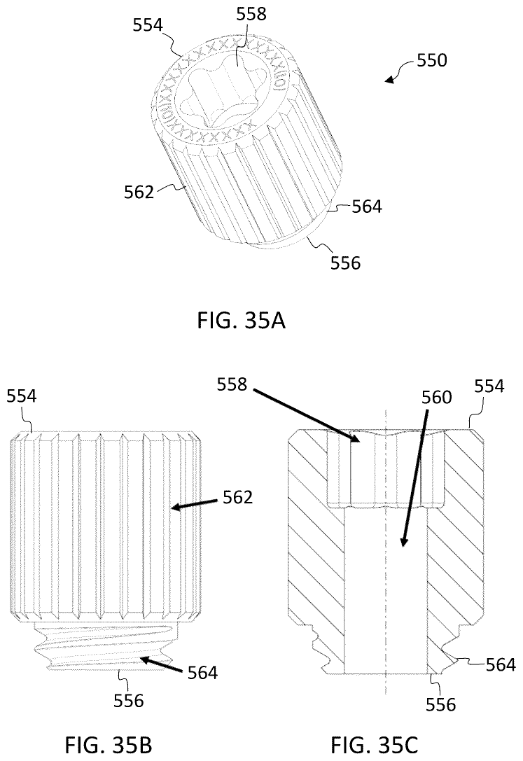

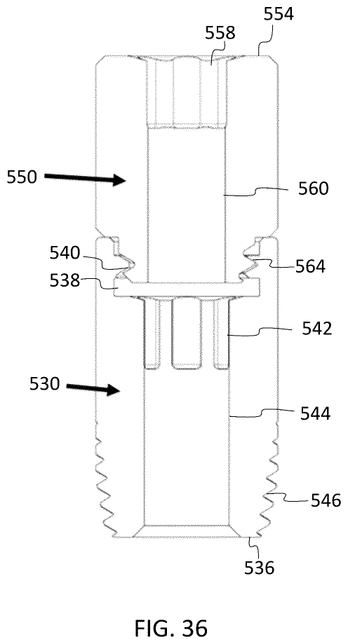

Turning now to FIGS. 35A-35C and 36, a K-wire sleeve 550 is shown according to one embodiment. The K-wire sleeve 550 accurately guides K-wires or guide wires to the trajectory of the underlying hole 18 in the plate 100A. The K-wire sleeve 550 may include a cylindrical body 552 extending from a proximal end 554 to a distal end 556 with a central cannulation.

As shown in FIG. 35C, the K-wire sleeve 550 defines a drive recess 558 and a K-wire cannulation 560 along the central tool axis. The drive recess 558 is located in the top of the sleeve 550 and may include a six-point star-shaped pattern, hexalobular profile, torx profile (e.g., T8), or other suitable recess. The drive recess 558 is suitable for engaging a driver instrument that facilitates insertion and removal of the sleeve 550. In one embodiment, the drive recess 558 in the sleeve 550 is the same as the drive recess 542 in the drill guide 530. In this manner, the drive recess 558 allows for quick removal of the sleeve 550 using the same driver as offered in the rest of the set. The K-wire sleeve 550 is cannulated 560 for accurate targeting of K-wires or guide wires through the monoaxial plate threaded holes 18. The inner diameter 560 of the sleeve 550 accurately locates guide wires or K-wires to the trajectory of the underlying monoaxial hole 18 in the plate 100A.

An exterior surface of the K-wire sleeve 550 may have a textured surface 562, which may be gripped by the user. For example, the sleeve 550 may have a knurled outer diameter to allow for fast hand-removal, if desired. As shown in FIG. 35B, the sleeve 550 may have a straight knurl on the outer diameter or other suitable configuration for optional hand removal. The distal end 556 includes a threaded portion 564 configured to lock into the top of the threaded drill guide 530. As best seen in FIG. 36, the K-wire sleeve 550 attaches to the monoaxial locking drill guide 530 by threading threaded portion 564 into the internal thread 540 of the drill guide 530. The K-wire sleeve 550 is aligned with the locking drill guide 530 along the central longitudinal tool axis, such that the inner cannulations 544, 560 are aligned. The drill guide 530 may be threaded into the monoaxial fastener hole 18 via tapered thread 546, thereby providing a trajectory for the K-wire to the underlying hole 18 in the plate 100A.

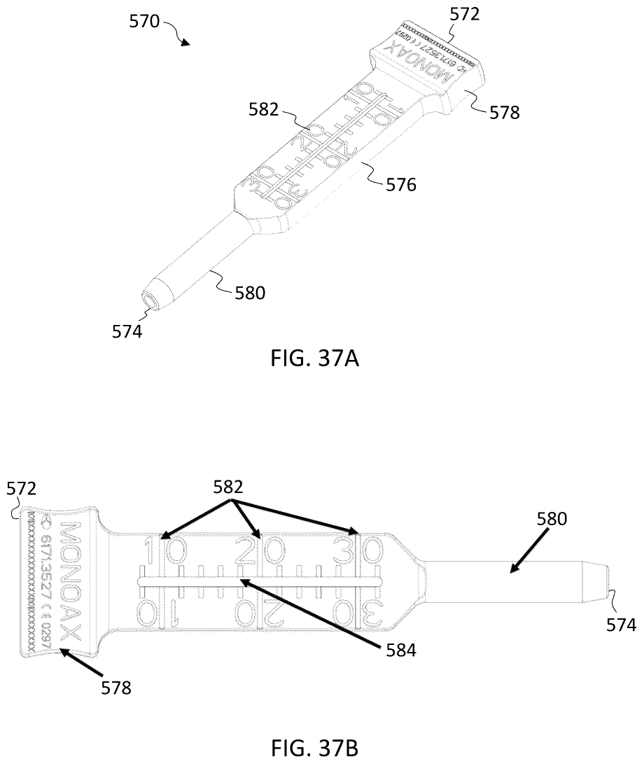

Turning now to FIGS. 37A-37B, a calibrated measuring block 570 for the monoaxial volar targeting guide 670 is shown according to one embodiment. The measuring block 570 is configured to provide a calibrated measurement option when drilling holes through the monoaxial volar targeting guides 670 in the monoaxial plates 100A. The measuring block 570 may have a cannulated body extend from a proximal end 572 to a distal end 574 along a central tool axis. The measuring block 570 may include a rectangular block face 576 with an enlarged grip 578 and a cylindrical distal tip 580.

A series of calibrated etches 582 or other markings and an elongated viewing window 584 may be located on the rectangular block face 576. The rectangular block face 576 may include calibrated etches 582, for example, with etched lines and numbers or other suitable markings or indicators. In one embodiment, the calibrated etching 582 may include etched lines spaced 2 mm apart, for example, from 8 mm to 32 mm to encompass the range of screw sizes offered in the distal radius set. The viewing window 584 may include a longitudinal slit or cut that runs along the long axis of the block 570. The proximal etch 638 on the calibrated drill 630 may be lined up with the corresponding calibrated etches 582 through the viewing window 584 in order to determine screw size for a given drilled hole.

The enlarged grip 578 may have a contoured grip surface, which is indented at the proximal end 572 to provide an ergonomic surface for surgeons to grip. The distal tip 580 may have a long tip length for drill guide compatibility. The distal tip 580 may have a cylindrical body that terminates at a tapered distal end 574. The long tip 580 of the measuring block 570 is configured to fit through the holes on the monoaxial volar targeting guide 670. The length also ensures that the distal calibrated etches 640, 642 on the calibrated drill 630 are not visible while using the proximal etch 638 on the calibrated drill 630 for measurement. A longer tip 580 also reduces potential for K-wire interference above the plate 100A.

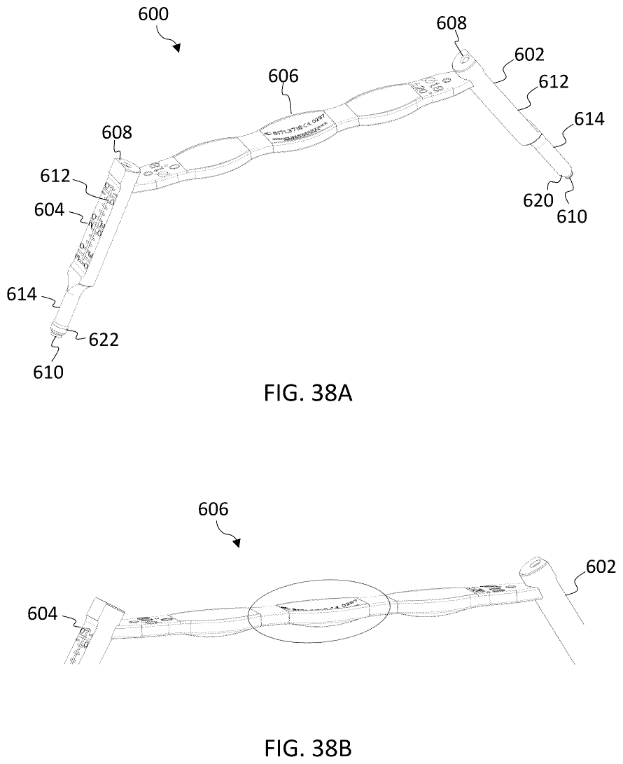

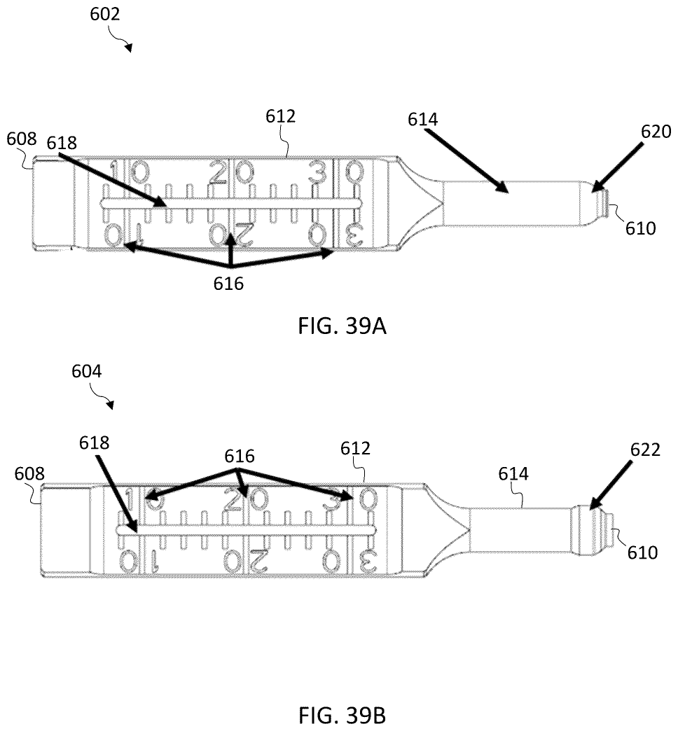

Turning now to FIGS. 38A-39B, a calibrated polyaxial drill guide 600 is shown according to one embodiment. The calibrated drill guide 600 may measure drilled depth at monoaxial and/or polyaxial trajectories in plate holes 16, 18. The calibrated polyaxial drill guide 600 is similar to the calibrated measuring block 570 with measuring blocks 602, 604 on opposite sides of a handle 606. One measuring block 602 is configured to guide up to 20 degrees of polyaxial trajectories and the opposite measuring block 604 is configured to provide a zero degree nominal trajectory guide.

As best seen in FIGS. 39A-39B, the calibrated measuring blocks 602, 604 each have a cannulated body extend from a proximal end 608 to a distal end 610 along a central tool axis. The measuring blocks 602, 604 may include a rectangular block face 612 with a cylindrical distal tip 614. A series of calibrated etches 616 or other markings and an elongated viewing window 618 may be located on the rectangular block face 612. The viewing window 618 may include a longitudinal slit or cut that runs along the long axis of the block 612. The calibrated etches 616 may include etched lines spaced 2 mm apart, for example, from 08 mm to 32 mm to encompass the range of screw sizes offered in the distal radius set. The proximal etch 638 on the calibrated drill 630 is configured to line up with the corresponding calibrated etches 616 through the viewing window 618 in order to determine screw size for a given drilled hole.

The distal tip 614 may have a cylindrical body that is compatible with a targeting guide. For example, the tip of the nominal end of the drill guide 600 is configured to fit through a polyaxial targeting guide. The tip lengths of the calibrated drill guide 600 ensure that the distal calibrated etches 640, 642 on the calibrated drill 630 are not visible while using the proximal etch 638 on the calibrated drill 630 for measurement. The drill guide 600 includes different tips 614 on each side. As best seen in FIG. 39A, measuring block 602 includes a polyaxial tip 620, which provides+/−20 degrees of polyaxial positioning. The polyaxial tip 620 may include a tapered or spherical tip configured to fit in the polyaxial plate holes 16. As best seen in FIG. 39B, measuring block 604 includes a monoaxial or nominal angle tip 622, which provides a zero-degree nominal trajectory. The nominal angle tip 622 may include a rim or band around its circumference. The rim may be conical, spherical, tapered, or otherwise configured to fit in the plate holes 16, 18 to maintain the nominal trajectory. These guides 602, 604 are located on opposite sides of the handle 606 and may be denoted with laser marks 616 or other suitable markings or indicators for clarity.

As best seen in FIG. 38B, the contoured handle 606 provides a secure way for holding the drill guide 600 during procedures. The ergonomic handle 606 may include a scalloped and contoured handle, which allows for comfortable grip of the drill guide 600 and prevents slipping when in a surgical environment. The ends of the handle 606 connect to the proximal ends 608 of the respective measuring blocks 602, 604. The measuring blocks 602, 604 may be angled outward from the handle 606 such that the distal tips 610 angle outward and away from the center. The calibrated polyaxial drill guide 600 is configured to ensure precise drilling angles and depths. The calibrated drill guide 600 may be useful to measure drilled depth at monoaxial trajectories and/or polyaxial trajectories in the plate holes 16, 18.

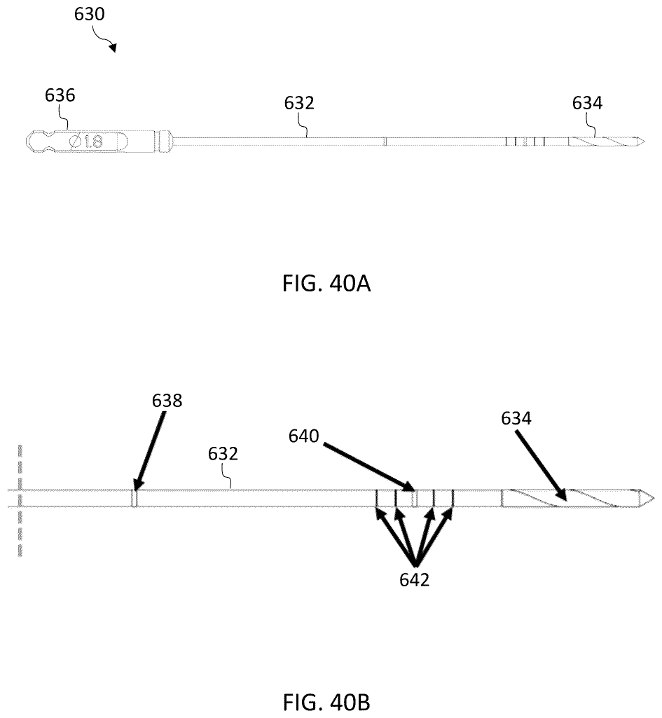

Turning now to FIGS. 40A-40B, a calibrated drill bit 630 is shown according to one embodiment. The drill bit 630 includes a shaft 632 with cutting flutes 634 at the distal end and an attachment portion 636 at the proximal end. The cutting flutes 634 may include a cutting edge configured to efficiently remove bone and debris as the hole is formed. In one embodiment, the cutting flutes 634 may have a shorter flute length (e.g., 16 mm) configured to reduce the potential for soft tissue irritation during surgery and to allow for distal laser marks. The attachment portion 636 may include an attachment interface to connect with a powered drill or other suitable instrument.

The drill bit 630 includes primary laser etches 638, 640 and secondary laser etches 642. As best seen in FIG. 40B, the shaft 632 may include two thick primary laser etches 638, 640 and four thin secondary laser etches 642. The laser etches 638, 640, 642 may be provided in two groups: a single proximal laser etch 638, and five distal laser etches 640, 642. The primary proximal and distal laser etches 638, 640 may include a pair of etches and the secondary distal etches 642 may include single etches at equal intervals denoting screw size. Although laser etches are exemplified, it will be appreciated that other suitable markings or indicators may also be used.

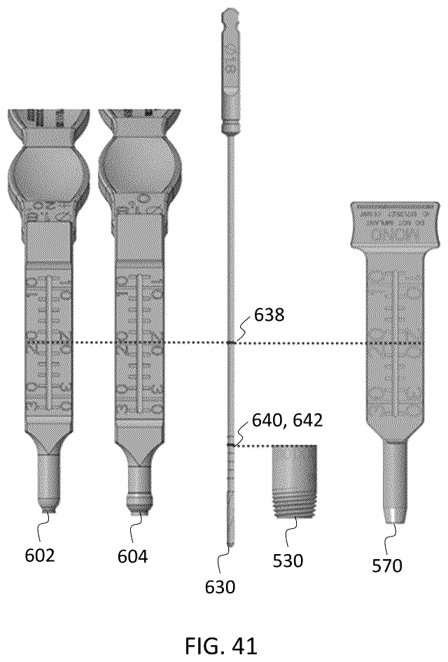

As best seen in FIG. 41, the calibrated drill 630 is configured to be compatible with four different measurement options. The proximal laser etch 638 is configured to measure using both sides 602, 604 of the calibrated drill guide 600 and the monoaxial measuring block 570. When the drill bit 630 is positioned through the measuring blocks 570, 602, 604, the user is able to see the proximal etch 638 through the window 584, 618 and read the calibrated markings 582, 616 on the measuring block 570, 602, 604. The distal laser etches 640, 642 are configured to measure with the monoaxial locking drill guide 530. The distal etches 640, 642 align with the proximal end 534 of the drill guide 530 to determine the measurement. The components form a one drill system where the measuring instruments 530, 570, 602, 604 are interchangeable to determine screw size for a given drilled hole.

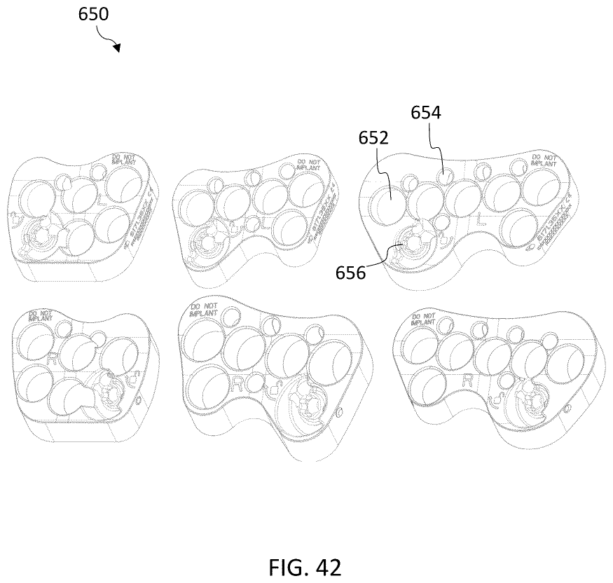

Turning now to FIG. 42, a polyaxial volar targeting guide family 650 is shown according to one embodiment. The targeting guides 650 may include narrow, standard, and wide sizes for both left and right plates. The polyaxial volar targeting guides 650 may include varying outer contours to roughly match the underlying contour of the mating polyaxial plate geometry so the surgeon's view is not blocked by instrumentation. The polyaxial volar targeting guides 650 may include a cam lock screw 656 which fixes the guide 650 to the proximal radial hole in their respective plate 100.

Each targeting guide 650 may include a plurality of through openings 652, which correspond to each of the respective fastener openings 16 in the plate 100. The openings 652 may be configured in order to drill the pilot holes at the appropriate trajectories for each fastener opening 16, and subsequently receive the respective fasteners 12 at the correct trajectories. The targeting guide 650 may also include a plurality of K-wire openings 654 which match with the K-wire openings 20 in the plate 100. The targeting guide 650 may be secured to the plate 100 with one or more fasteners or may be secured to the plate 100 through an integrated connection system such as a thumb screw, an interference fit, etc.

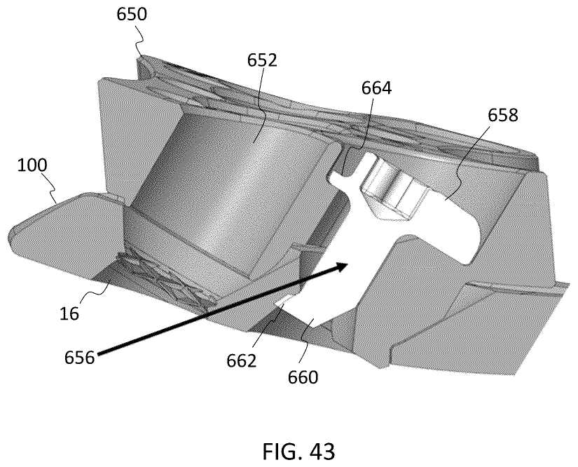

In one embodiment, the targeting guide 650 may be secured to the plate 100 with a cam lock 656. As best seen in FIG. 43, a cross section of cam lock 656 depicts the interference fit with the polyaxial hole cone geometry. The cam lock 656 may include a screw with a head 658 and shaft 660 extending from the head 658. The shaft 660 includes a radially extending flange 662 extending from the distal end of the shaft 660. The head 658 also includes a cam surface 664 that extends radially from the head 658. When rotated, the flanges 662, 664 may engage corresponding recesses on the plate 100, thereby locking the targeting guide 650 to the plate 100. Additional details of the cam lock and targeting guides are provided in U.S. Pat. No. 11,331,128, which is incorporated by reference herein in its entirety for all purposes.

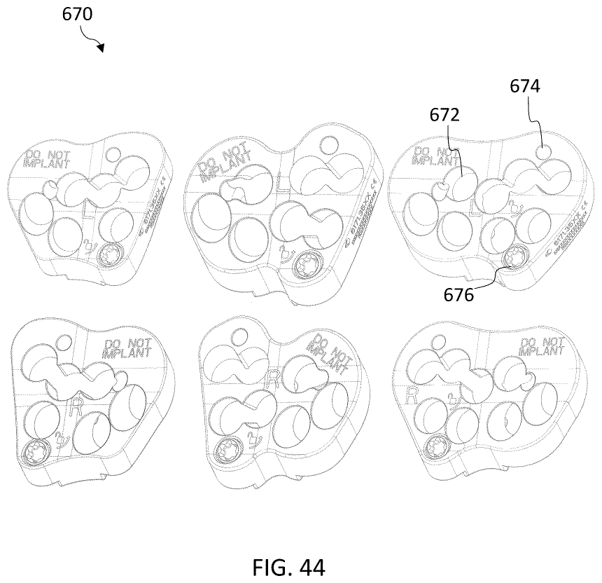

Turning now to FIG. 44, a monoaxial volar targeting guide family 670 is shown according to one embodiment. The monoaxial volar targeting guide 670 is similar to the polyaxial targeting guide 650 except the cam lock is replaced with a locking screw attachment. The polyaxial volar targeting guides 670 may come in narrow, standard, and wide sizes for both left and right plates. Each targeting guide 670 may include cannulated openings or through openings 672, which correspond to each of the respective fastener openings 18 in the plates 100A and guide wire or K-wire openings 674 which match with the guide wire or K-wire openings 20 in the plate 100A. The through openings 672 and/or guide wire openings 674 may be overlapping in some instances for the desired trajectories.

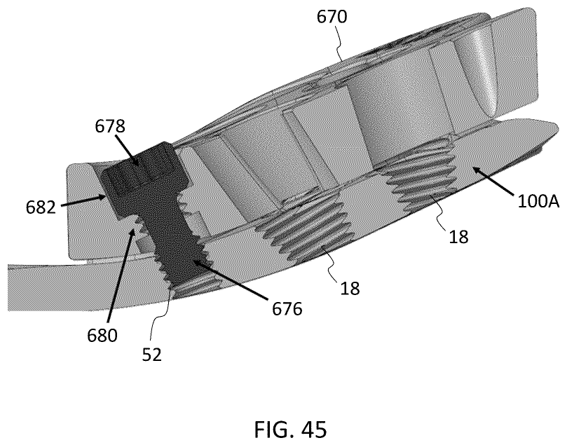

The targeting guide 670 may be secured to the plate 100A with locking screw 676. The screw 676 may include a drive recess 678 (e.g., a T8 recess) in its head for compatibility with a driver instrument in the distal radius set. The shaft of the screw 676 threads into a self-retaining threaded recess 680 in the targeting guide 670. Once rotated into position, the self-retaining threaded attachment screw 676 locks into the dedicated threaded hole 22 in the mid-shaft strut of the monoaxial distal radius plate 100A. The threaded shaft of the locking screw 676 threads into threaded opening 22 in the plate 100A, thereby fixing the targeting guide 670 to the plate 100A. When fully seated as shown in FIG. 45, the screw 676 may sit in a counterbore 682 on the targeting guide 670 so that it is out of the way of drilling operations during surgery.

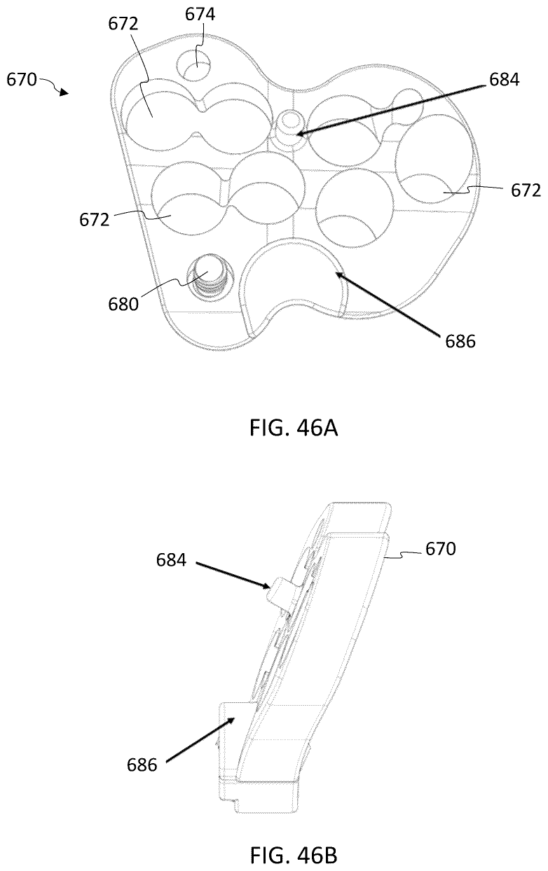

As shown in FIGS. 46A-46B, the bottom of the monoaxial volar targeting guide 670 may include additional attachment and aligning features. In one embodiment, the monoaxial volar targeting guide 670 includes a K-wire hole boss 684 and/or a graft hole boss 686, which protrude from the bottom surface of the guide 670. The K-wire and graft window bosses 684, 686 may help to align the targeting guide 670 to the monoaxial plate 100A. Both the polyaxial and monoaxial volar targeting guides 650, 670 offer a time-efficient solution for drilling holes in the head of the polyaxial and monoaxial distal radius plates 100, 100A at nominal trajectories.

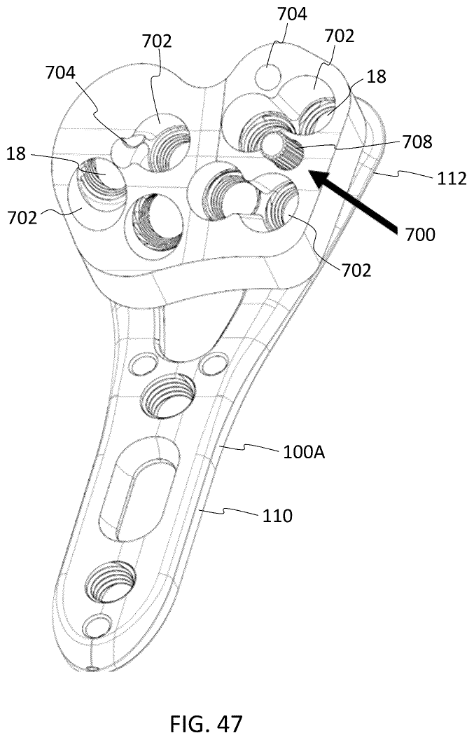

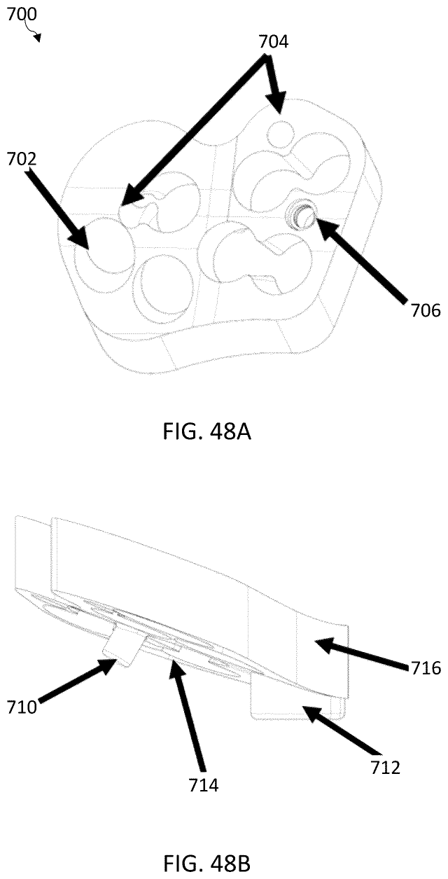

Turning now to FIG. 47, a monoaxial backpack or monoaxial guide 700 is shown attached to a monoaxial plate 100A according to one embodiment. As best seen in FIG. 48A, similar to guides 650, 670, the monoaxial backpack 700 includes coaxial through-holes 702 aligned with fastener openings 18 for accommodating a calibrated drill guide, through-holes 704 to accommodate K-wires, and a threaded hole 706 to retain an attachment screw 708. The hole trajectories for fastener through-holes 702 coaxially align with the monoaxial screw holes 18 in the plate 100A and are configured to accommodate a calibrated drill guide. Similarly, the K-wire through-holes 704 coaxially align with the K-wire holes 20 in the plate 100A. The retaining hole 706 retains the attachment screw 708 in the volar targeting guide 700.

As best seen in FIG. 48B, the guide 700 may also include an K-wire alignment boss 710 and a graft window cam 712, which prevent rotation of the targeting guide 700. The alignment boss 710 may be configured to seat within the central K-wire opening 20 in the monoaxial plate 100A to prevent rotation of the volar targeting guide 700 when attached. The graft window cam 712 sits partially in the graft window 50 of the distal radius volar plate 100A to rigidly align the volar targeting guide 700. The under surface or bottom surface 714 of the targeting guide 700 may be contoured to match the top surface 106 of the monoaxial plate 100A for fit. The body is low profile 716 to allow the surgeon to work in the anatomical site without obstruction.

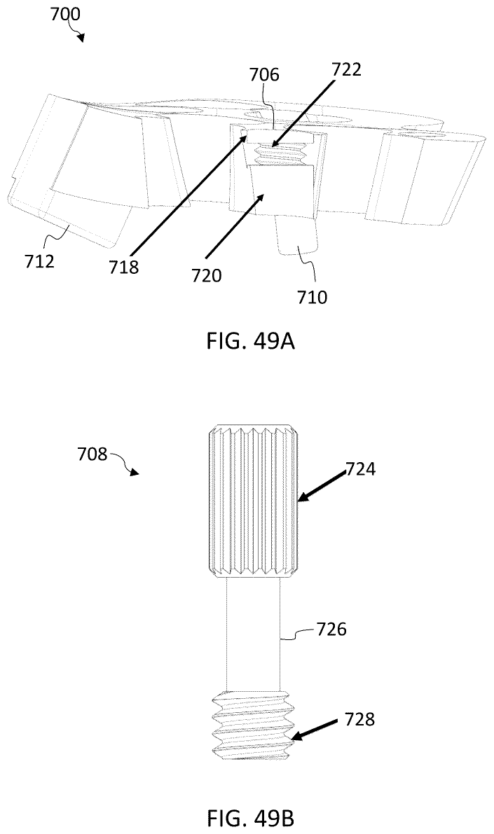

The volar targeting guide 700 may be locked by hand to the plate using a partially threaded attachment screw 708. The screw 708 is retained in the threaded hole 706 in the volar targeting guide 700, and it threads into the dedicated hole 52 in the monoaxial plate 100A. As best seen in the cross-section shown in FIG. 49A, the retaining hole 706 includes an upper counterbore 718 to create a parallel mating surface for the bottom of the attachment screw head. The retaining hole 706 also includes a lower counter bore 720 to accommodate the attachment screw shank. The partially threaded inner diameter allows the mating screw 708 to be retained in the volar targeting guide 700 before attaching to the monoaxial plate 100A.

As best seen in FIG. 49B, the attachment screw 708 includes a head 724 and a shaft 726 with threads 728 at the distal end. The attachment screw 708 may include a knurled elongated head 724 for hand-operation. The attachment screw 708 may include a partially threaded shaft 726 with a threaded profile 728 configured to be retained in the volar targeting guide 700 by the threaded section 722 in the guide 700. The screw 708 then attaches to the dedicated hole 22 in the monoaxial plate 100A.

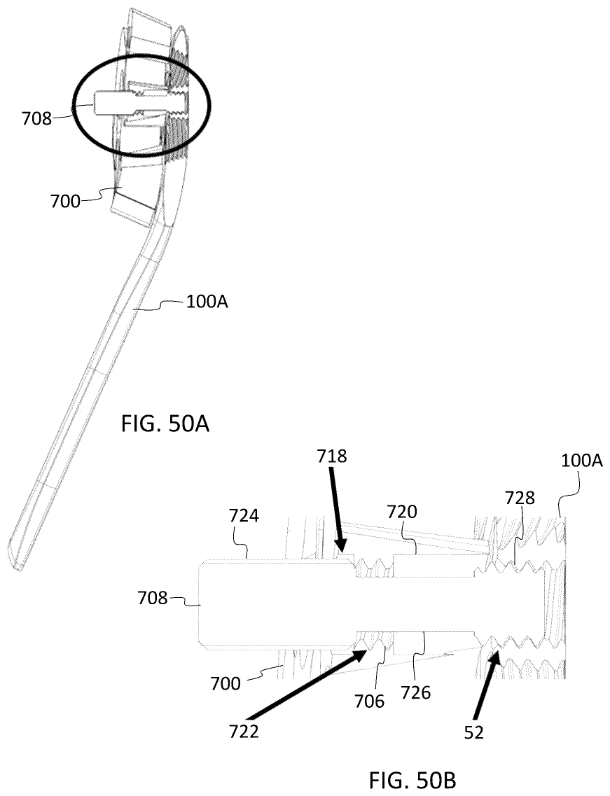

As best seen in FIGS. 47, the attachment screw 708 seats into the volar targeting guide 700 without interfering with the surrounding holes 18 or the drilling steps. The surgeon can easily remove the targeting guide 700 once the drilling has been completed. As shown in the assembly cross-section shown in FIGS. 50A-50B, the attachment screw 708 seats into the counterbore 718 of the retaining hole 706. The threaded portion 728 of the attachment screw 708 threads into the dedicated hole 52 in the monoaxial plate 100A, thereby temporarily securing the backpack 700 to the plate 100A. The volar targeting guide is attached to plate 100A at a discreet location to allow for easy access to the surrounding holes 18. In addition, the attachment hole 706 is aligned with the ulnar side 130 of the distal radius plate 100A, which allows for other devices to be attached using the same dedicated hole 52, such as a lunate facet hook, after using the targeting guide 700. The guide 700 provides for the speed of procedure and accurate monoaxial targeting while also offering the added functionality of the attachment hole interface.

It will be further understood that various changes in the details, materials, and arrangements of the parts which have been described and illustrated in order to explain the nature of this invention may be made by those skilled in the art without departing from the scope of the invention as expressed in the claims. One skilled in the art will appreciate that the embodiments discussed above are non-limiting. It will also be appreciated that one or more features of one embodiment may be partially or fully incorporated into one or more other embodiments described herein.

What is claimed is:

1. A distal radius bone plate comprising:
a body extending along a central longitudinal axis from a proximal end configured to sit on a shaft of a radius to a distal end configured to sit on a distal radius;
a top surface and an opposite, bottom surface configured to contact the radius; and
an elongated proximal shaft and an enlarged distal head extending therefrom, wherein the enlarged distal head defines a tendon groove in the distal end configured to accommodate a flexor pollicus longus tendon, the tendon groove includes a concave recess at a distal edge of the plate forming two rounded lobes on either side of the groove, the lobes include an ulnar-side lobe and a radial-side lobe.

2. The bone plate of claim 1, wherein the two rounded lobes are not symmetrical.

3. The bone plate of claim 1, wherein the ulnar-side lobe is longer than the radial side lobe.

4. The bone plate of claim 1, wherein the tendon groove is located off-center of the central longitudinal axis of the plate.

5. The bone plate of claim 1, wherein the radial-side lobe is thicker than the ulnar-side lobe.

6. The bone plate of claim 1, wherein the ulnar-side lobe includes a curved ulnar surface on the bottom surface of the plate to promote best fit of the distal head with the distal radius.

7. The bone plate of claim 1, wherein the ulnar-side lobe includes a distal ulnar undercut through a distal-most portion of the ulnar-side lobe.

8. The bone plate of claim 1, wherein the ulnar-side lobe includes a variable chamfered surface along its edge to minimize tendon disruption.

9. A stabilization system for stabilizing a distal radius, the system comprising:
a plate having a top surface and an opposite, bottom surface configured to contact a radius, the plate has an elongated proximal shaft and an enlarged distal head extending therefrom, the distal head defines a tendon groove configured to receive a flexor pollicus longus tendon, the tendon groove includes a concave recess at a distal edge of the plate forming two rounded lobes on either side of the groove, the lobes include an ulnar-side lobe and a radial-side lobe each including fastener openings, and the ulnar-side lobe is longer than the radial side lobe; and
a plurality of fasteners receivable through the fastener openings and configured to lock the plate to the distal radius.

10. The system of claim 9, wherein the fastener openings include polyaxial openings having a cone of angulation up to 40 degrees.

11. The system of claim 9, wherein the radial-side lobe defines a first opening with a trajectory targeted at a radial styloid.

12. The system of claim 9, wherein the lobes define a distal-most row of fastener openings, and when the fasteners are received therein, screw trajectories of the distal-most row of fastener openings are aligned so that nominal trajectories of the fasteners follow articular surfaces of a radiocarpal joint and a distal radio-ulnar joint.

13. The system of claim 12, wherein the lobes define a second row of fastener openings, and when the fasteners are received therein, screw trajectories of the second row of fastener openings have trajectories that fit between the distal-most row of fasteners such that the trajectories converge with the distal-most row screw trajectories.

14. The system of claim 13, wherein the second row of fastener openings include a first opening in the radial-side lobe and a second fastener opening in the ulnar-side lobe, the first opening receives a fastener that extends dorsally and toward the radial styloid screw trajectory and the second opening receives a fastener that extends dorsally and fits between trajectories of the distal-most openings on the ulnar-side lobe.

15. The system of claim 9, wherein the proximal shaft defines a positioning slot configured to receive a non-locking fastener such that the plate is adjustable in proximal-distal and/or medial-lateral directions during provisional placement of the plate.

16. A method of installing a distal radius plate comprising:
providing a distal radius plate having an elongated proximal shaft and an enlarged distal head extending therefrom, the distal head defines a tendon groove having a concave recess at a distal edge of the plate forming two rounded lobes on either side of the groove, the lobes include an ulnar-side lobe and a radial-side lobe each defining fastener openings and guide wire openings therethrough, the proximal shaft defines a positioning slot, fastener openings, and guide wire openings therethrough;
provisionally placing the plate against a distal radius by inserting a non-locking screw through the positioning slot and moving the plate in proximal-distal and/or medial-lateral directions to center the plate on the distal radius;
inserting one or more guide wires through the guide wire openings to hold the plate in position and/or use as a guide for inserting fasteners; and
inserting fasteners through the fastener openings in the radial-side lobe including one fastener into a radial styloid and one fastener following a radiocarpal joint, and in the ulnar-side lobe including one fastener following the radiocarpal joint and one fastener following a distal radio-ulnar joint.

17. The method of claim 16, wherein the plate defines a second row of fastener openings, and the method further includes inserting fasteners through the second row of fastener openings including one fastener in the radial-side lobe that extends dorsally toward the radial styloid fastener and one fastener in the ulnar-side lobe that extends dorsally and fits between the radiocarpal joint and distal radio-ulnar joint fasteners in the ulnar-side lobe.

18. The method of claim 16, further comprising independently adjusting the radial-side and ulnar-side lobes intraoperatively to optimize placement against the distal radius.

19. The method of claim 16, wherein the fastener openings are polyaxial holes and the fasteners are variable angle fasteners providing for variable angle screw insertion with up to 40 degrees of cone of angulation.

20. The method of claim 16, wherein the fastener openings are monoaxial holes and the fasteners are fixed angle fasteners providing for fixed angle screw insertion.
